(19) United States
(12) Patent Application Publication
(10) Pub. No.: US 2018/0301632 A1
(43) Pub. Date: Oct. 18, 2018

(54) SPIRALLY CONFIGURED CIS-STILBENE/FLUORENE HYBRID MATERIAL AND ORGANIC ELECTROLUMINESCENT DEVICE USING THE SAME

(71) Applicant: JUST ABOUT SHOWING CO., LTD., New Taipei City (TW)

(72) Inventor: Jen-Cherng LEE, NEW TAIPEI CITY (TW)

(21) Appl. No.: 15/656,389

(22) Filed: Jul. 21, 2017

(30) Foreign Application Priority Data
Apr. 13, 2017 (TW) 106112304

(51) Int. Cl. H01L 51/00; C07C 211/61; C07C 255/52; C07D 239/26; C07C 255/61; C09K 11/06; C09K 11/02; C07C 13/72

(52) U.S. Cl. CPC H01L 51/0057; H01L 51/006; C07C 211/61; C07C 255/52; C07D 239/26; H01L 51/5206; C09K 11/06; H01L 51/0067; C09K 11/025; H01L 51/0085; C07C 13/72; C07C 255/61

(57) ABSTRACT

A spirally configured cis-stilbene/fluorene hybrid material is shown in formula (1),

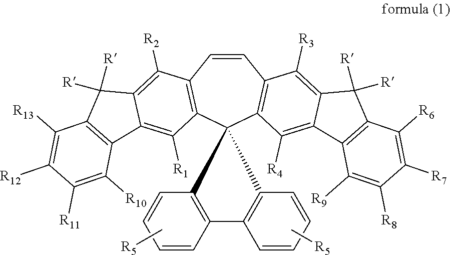

R′ is an alkyl group or represented by formula (2),

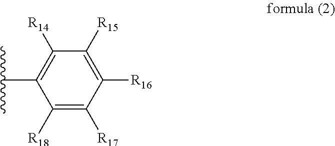

wherein R₁ to R₄, R₆, R₈ to R₁₁ and R₁₃ to R₁₈ are independently selected from the group consisting of hydrogen atom, halogen atom, cyano group, alkyl group, cycloalkyl group, alkoxy group, amino group, haloalkyl group, thioalkyl group, silyl group and alkenyl group, R₅ is a hydrogen atom, tert-butyl group or aryl group, R₇ and R₁₂ are independently selected from the group consisting of hydrogen atom, aryl group, diarylamino group, cyano group, heteroaryl (e.g., pyridine group, or pyrimidine group).

### CROSS REFERENCE TO RELATED APPLICATIONS

This Non-provisional application claims priority under 35 U.S.C. § 119(a) on Patent Application No(s). 106112304 filed in Taiwan, Republic of China on Apr. 13, 2017, the entire contents of which are hereby incorporated by reference.

### BACKGROUND OF THE INVENTION

### Field of Invention

The present invention relates to an organic electroluminescent material and an organic electroluminescent device using the same and, in particular, to a spirally configured cis-stilbene/fluorene hybrid material and the organic electroluminescent device using the same.

### Related Art

With the advances in electronic technology, a light weight and high efficiency flat display device has been developed. An organic electroluminescent device becomes the mainstream of the next generation flat panel display device due to its advantages of self-luminosity, no restriction on viewing angle, power conservation, simple manufacturing process, low cost, high response speed, full color and so on.

In general, the organic electroluminescent device includes an anode, an organic luminescent layer and a cathode. When applying a direct current to the organic electroluminescent device, holes and electrons are injected into the organic luminescent layer from the anode and the cathode, respectively. Charge carriers move and then transport into the organic luminescent layer because of the potential difference caused by an applied electric field. The resulting excited luminescent molecules (i.e., excitons) are generated and followed by the recombination of the electrons and the electron holes may lead to emission in the organic luminescent layer due to release the energy in the form of light.

Nowadays, the organic electroluminescent device usually adopts a host-guest emitter system. The organic luminescent layer disposed therein includes a host material and a guest material. The holes and the electrons are transmitted to the host material, and further transferred to the guest material to form excitons and then generate light.

In addition, regarding to materials which emit red, green or blue light (three primary colors of light), the energy gap of the material which emits a blue light is higher than that emits a red light or a green light. Therefore, the material which emits a blue light is usually a planar aromatic compound with a short conjugation fragment and simpler structure. Such material is usually thermally unstable, and the device comprising such material may have a short lifespan accordingly. Moreover, molecules having planar or nearly planar structures tend to stack to each other which may result to form an excimer, and may further affect colors of emission lights and luminous efficiencies. Therefore, when used in an organic electroluminescent device, such materials may only be doped to a host material. The value of application may therefore be limited.

Besides, the selection of organic electroluminescent material is not only based on the matching of HOMO and LUMO energy levels but also counts on the high decomposition temperature in order to avoid pyrolysis during thermal vacuum deposition and also thus avoid the decrease in thermal stability.

Accordingly, it is an urgent need to provide an organic electroluminescent material and an organic electroluminescent device using the same which have high luminous efficiency, and excellent thermal stability and film forming ability.

### SUMMARY OF THE INVENTION

In view of the foregoing objectives, the invention provides a spirally configured cis-stilbene/fluorene hybrid material and an organic electroluminescent device by using the same. The spirally configured cis-stilbene/fluorene hybrid material has high luminous efficiency, and excellent thermal stability and film forming ability.

To achieve the above objective, one embodiment of the invention discloses a spirally configured cis-stilbene/fluorene hybrid material, which comprises a structure of the following General Formula (1).

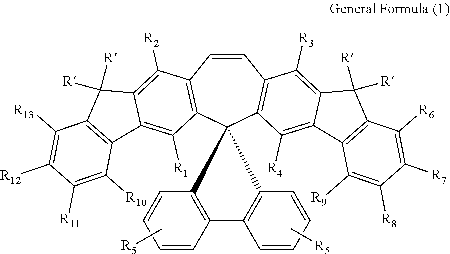

In the General formula (1), R′ is an alkyl group or represented by General Formula (2).

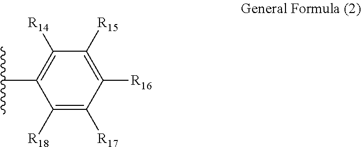

In the General formula (1), R₁ to R₄, R₆, R₈ to R₁₁ and R₁₃ to R₁₈ are each independently selected from the group consisting of a hydrogen atom, halogen atom, cyano group, alkyl group, cycloalkyl group, alkoxy group, amino group, haloalkyl group, thioalkyl group, silyl group and alkenyl group. R₅ a hydrogen atom, tert-butyl group or aryl group. R₇ and R₁₂ are each independently selected from the group consisting of a hydrogen atom, aryl group, diarylamine, cyano group and hetero aromatic ring (for example, a pyridine or a pyrimidine).

In one embodiment, the alkyl group is selected from the group consisting of a substituted or unsubstituted straight-chain alkyl group with the carbon number of 1 to 6, and a substituted or unsubstituted branched-chain alkyl group with the carbon number of 3 to 6. The cycloalkyl group is a substituted or unsubstituted cycloalkyl group with the carbon number of 3 to 6. The alkoxy group is selected from the group consisting of a substituted or unsubstituted straight-chain alkoxy group with the carbon number of 1 to 6, and a substituted or unsubstituted branched-chain alkoxy group with the carbon number of 3 to 6. The amino group is selected from the group consisting of secondary amino group and tertiary amino group. The secondary amino group is an amino group having one aromatic ring substituent or having one C1-C6 straight-chain alkyl substituent, branch-chain alkyl substituent, or non-aromatic ring substituent. The tertiary amino group is an amino group having two independent aromatic ring substituents or having two independent C1-C6 straight-chain alkyl substituents, branch-chain alkyl substituents, or non-aromatic ring substituents. The haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6. The thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6. The silyl group is selected from the group consisting of a substituted or unsubstituted straight-chain silyl group with the carbon number of 1 to 6, and a substituted or unsubstituted branched-chain silyl group with the carbon number of 3 to 6. The alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

In one embodiment, the spirally configured cis-stilbene/fluorene hybrid material is represented by following chemical formula (1), chemical formula (2), chemical formula (3), chemical formula (4a), chemical formula (4b), or chemical formula (5).

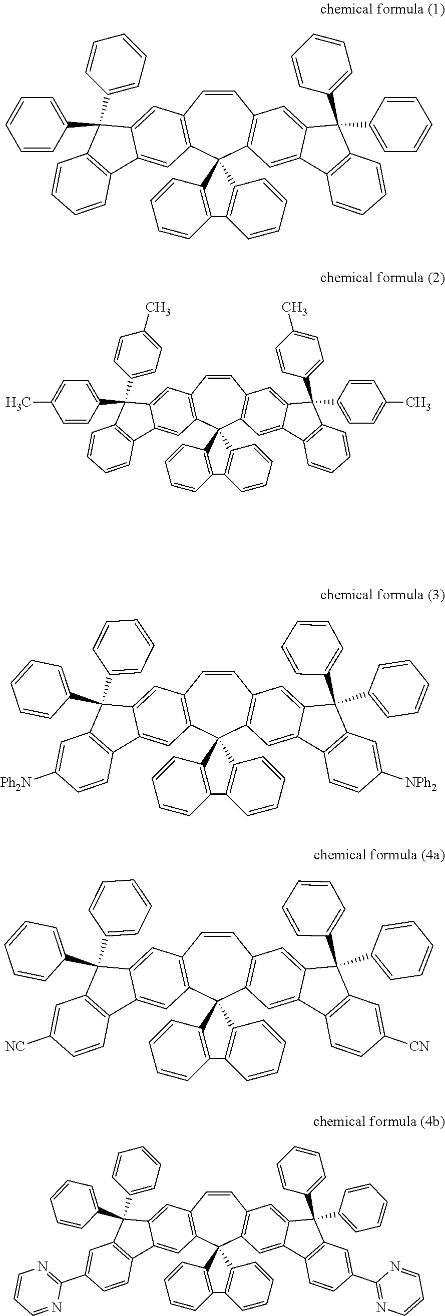

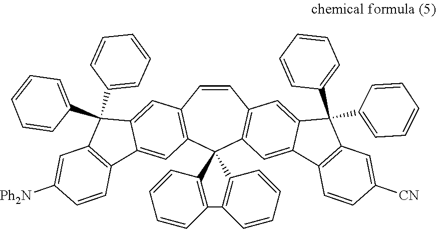

In one embodiment, the spirally configured cis-stilbene/fluorene hybrid material has glass transition temperatures ranged from 234° C. to 259° C.

In one embodiment, the spirally configured cis-stilbene/fluorene hybrid material has decomposition temperatures ranged from 469° C. to 492° C.

In one embodiment, the spirally configured cis-stilbene/fluorene hybrid material has oxidation potentials ranged from 0.33V to 1.03V and reduction potentials ranged from −1.77V to −2.13V.

In one embodiment, the spirally configured cis-stilbene/fluorene hybrid material has highest occupied molecular orbital energy levels (E_{HOMO}) ranged from −5.1 eV to −5.8 eV and lowest unoccupied molecular orbital energy levels (E_{LUMO}) ranged from −2.7 eV to −3.0 eV.

To achieve the above objective, an organic electroluminescent device is also disclosed. The organic electroluminescent device comprises a first electrode layer, a second electrode layer, and an organic luminescent unit. The organic luminescent unit is deposited between the first electrode layer and the second electrode layer. The organic luminescent unit has at least a spirally configured cis-stilbene/fluorene hybrid material as shown in General Formula (1).

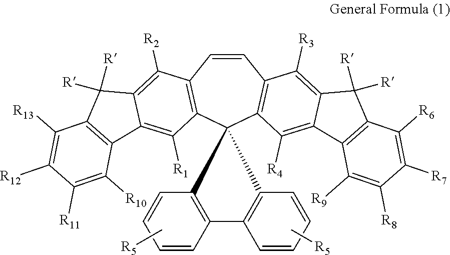

In the General formula (1), R′ is an alkyl group or represented by General Formula (2).

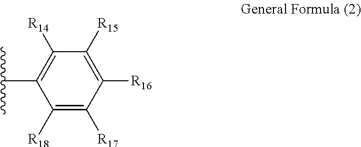

In the General formula (1), R₁ to R₄, R₆, R₈ to R₁₁ and R₁₃ to R₁₈ are each independently selected from the group consisting of a hydrogen atom, halogen atom, cyano group, alkyl group, cycloalkyl group, alkoxy group, amino group, haloalkyl group, thioalkyl group, silyl group and alkenyl group. R₅ is a hydrogen atom, tert-butyl group or aryl group. R₇ and R₁₂ are each independently selected from the group consisting of a hydrogen atom, aryl group, diarylamine, cyano group and hetero aromatic ring (for example, a pyridine and a pyrimidine).

In one embodiment, the alkyl group is selected from the group consisting of a substituted or unsubstituted straight-chain alkyl group with the carbon number of 1 to 6, and a substituted or unsubstituted branched-chain alkyl group with the carbon number of 3 to 6. The cycloalkyl group is a substituted or unsubstituted cycloalkyl group with the carbon number of 3 to 6. The alkoxy group is selected from the group consisting of a substituted or unsubstituted straight-chain alkoxy group with the carbon number of 1 to 6, and a substituted or unsubstituted branched-chain alkoxy group with the carbon number of 3 to 6. The amino group is selected from the group consisting of secondary amino group and tertiary amino group. The secondary amino group is an amino group having one aromatic ring substituent or having one C1-C6 straight-chain alkyl substituent, branch-chain alkyl substituent, or non-aromatic ring substituent. The tertiary amino group is an amino group having two independent aromatic ring substituents or having two independent C1-C6 straight-chain alkyl substituents, branch-chain alkyl substituents, or non-aromatic ring substituents. The haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6. The thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6. The silyl group is selected from the group consisting of a substituted or unsubstituted straight-chain silyl group with the carbon number of 1 to 6, and a substituted or unsubstituted branched-chain silyl group with the carbon number of 3 to 6. The alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

In one embodiment, the spirally configured cis-stilbene/fluorene hybrid material is represented by following chemical formula (1), chemical formula (2), chemical formula (3), chemical formula (4a), chemical formula (4b), or chemical formula (5).

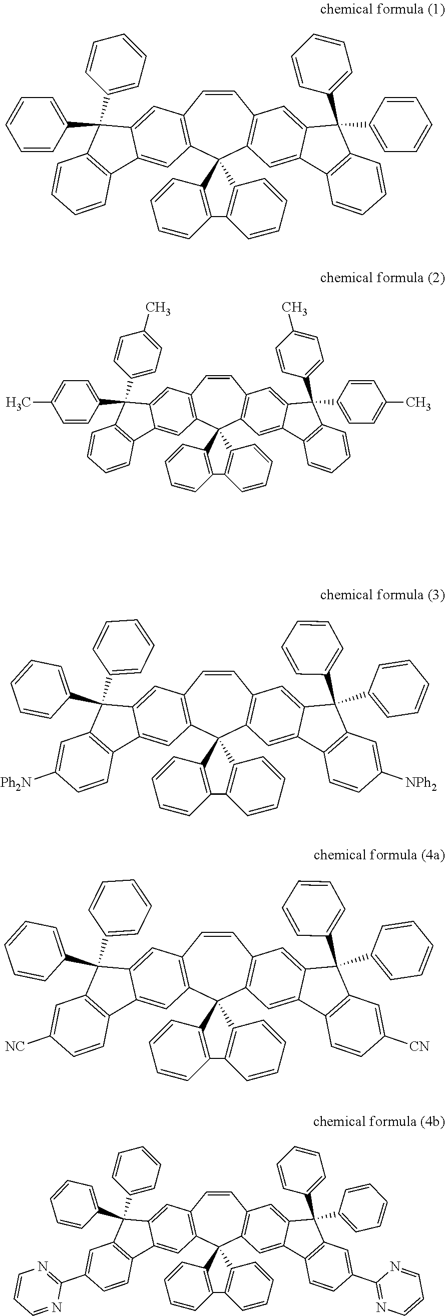

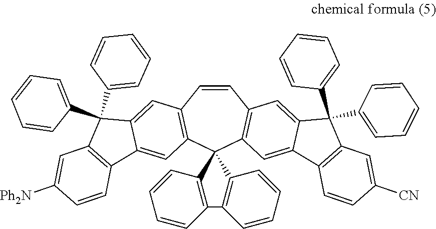

In one embodiment, the organic luminescent unit comprises an organic luminescent layer.

In one embodiment, the organic luminescent unit further comprises a hole transport layer and an electron transport layer, and the organic luminescent layer is deposited between the hole transport layer and the electron transport layer.

In one embodiment, the organic luminescent unit further comprises a hole injection layer, a hole transport layer, an electron transport layer and an electron injection layer, and the hole transport layer, the organic luminescent layer and the electron transport layer are sequentially deposited between the hole injection layer and the electron injection layer.

In one embodiment, the organic luminescent layer comprises the spirally configured cis-stilbene/fluorene hybrid material.

In one embodiment, the organic luminescent layer comprises a host material and a guest material, and the host material comprises the spirally configured cis-stilbene/fluorene hybrid material.

As mentioned above, in the spirally configured cis-stilbene/fluorene hybrid material and the organic electroluminescent device by using the same according to some embodiments of the present invention, the cis-stilbene fragment and the fluorine are doubly ortho-linked at C9 position, such that the cis-stilbene fragment is fixed and prevented from isomerization. In addition, two indeno templates are fused with the spirally configured cis-stilbene/fluorene hybrid system at C2-C3 and C7-C8 positions, and the resulting compound has a sterically-hindered structure so as to prevent the fluorescence quenching effect caused by p-p stacking. Moreover, the confined cis-stilbene has high fluorescence quantum effect. When the cis-stilbene is incorporated with the spirally configured fluorine and diindeno-fused system, the thermal stability and the film forming ability of the whole material can be further increased. Therefore, the resulting spirally configured cis-stilbene/fluorene hybrid material is suitable for an organic electroluminescent device.

### BRIEF DESCRIPTION OF THE DRAWINGS

The embodiments will become more fully understood from the detailed description and accompanying drawings, which are given for illustration only, and thus are not limitative of the present invention, and wherein:

FIG. 1 is a cross-sectional schematic diagram of an organic electroluminescent device of the second embodiment according to the invention;

FIG. 2 is a cross-sectional schematic diagram of an organic electroluminescent device of the third embodiment according to the invention; and

FIG. 3 is a cross-sectional schematic diagram of an organic electroluminescent device of the fourth embodiment according to the invention.

### DETAILED DESCRIPTION OF THE INVENTION

The embodiments of the invention will be apparent from the following detailed description, which proceeds with reference to the accompanying drawings, wherein the same references relate to the same elements.

Spirally Configured Cis-Stilbene/Fluorene Hybrid Material

A spirally configured cis-stilbene/fluorene hybrid material according to the first embodiment of the present invention has a structure of the following General Formula (1).

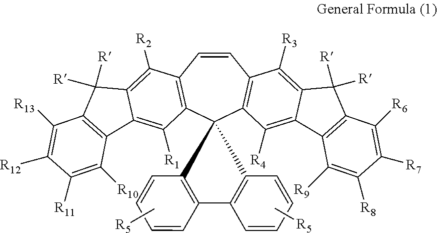

In the General formula (1), R′ is an alkyl group or represented by General Formula (2).

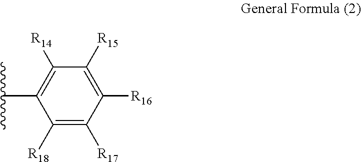

In the General formula (1), R₁ to R₄, R₆, R₈ to R₁₁, and R₁₃ to R₁₈ are each independently selected from the group consisting of a hydrogen atom, halogen atom, cyano group, alkyl group, cycloalkyl group, alkoxy group, amino group, haloalkyl group, thioalkyl group, silyl group and alkenyl group. R₅ is a hydrogen atom, tert-butyl group or aryl group. R₇ and R₁₂ are each independently selected from the group consisting of a hydrogen atom, aryl group, diarylamine, cyano group and hetero aromatic ring (for example, a pyridine or a pyrimidine).

In the present embodiment, the alkyl group can be selected from the group consisting of a substituted or unsubstituted straight-chain alkyl group with the carbon number of 1 to 6, and a substituted or unsubstituted branched-chain alkyl group with the carbon number of 3 to 6. The cycloalkyl group can be a substituted or unsubstituted cycloalkyl group with the carbon number of 3 to 6. The alkoxy group can be selected from the group consisting of a substituted or unsubstituted straight-chain alkoxy group with the carbon number of 1 to 6, and a substituted or unsubstituted branched-chain alkoxy group with the carbon number of 3 to 6. The amino group can be selected from the group consisting of secondary amino group and tertiary amino group. The haloalkyl group can be selected from the group consisting of a substituted or unsubstituted straight-chain haloalkyl group with the carbon number of 1 to 6, and a substituted or unsubstituted branched-chain haloalkyl group with the carbon number of 3 to 6. The thioalkyl group can be selected from the group consisting of a substituted or unsubstituted straight-chain thioalkyl group with the carbon number of 1 to 6, and a substituted or unsubstituted branched-chain thioalkyl group with the carbon number of 3 to 6. The silyl group can be selected from the group consisting of a substituted or unsubstituted straight-chain silyl group with the carbon number of 1 to 6, and a substituted or unsubstituted branched-chain silyl group with the carbon number of 3 to 6. The alkenyl group can be selected from the group consisting of a substituted or unsubstituted straight-chain alkenyl group with the carbon number of 2 to 6, and a substituted or unsubstituted branched-chain alkenyl group with the carbon number of 3 to 6.

Moreover, the secondary amino group can be an amino group having one aromatic ring substituent (for example, a phenyl amino group) or having one C1-C6 straight-chain alkyl substituent, branch-chain alkyl substituent, or non-aromatic ring substituent (for example, a methyl amino group). The tertiary amino group can be an amino group having two independent aromatic ring substituents (for example, a diphenyl amino group, —NPh₂) or having two independent C1-C6 straight-chain alkyl substituents, branch-chain alkyl substituents, or non-aromatic ring substituents (for example, a dimethyl amino group).

The spirally configured cis-stilbene/fluorene hybrid material according to the present embodiment represented by General Formula (1) can be a material of an organic luminescent layer in an organic electroluminescent device, especially can be a host material. An example is the compound of chemical formula (1), Bis-BnCPSTIF-1, where R′ is represented by General Formula (2), and R₁ to R₁₈ are all independent hydrogen atoms.

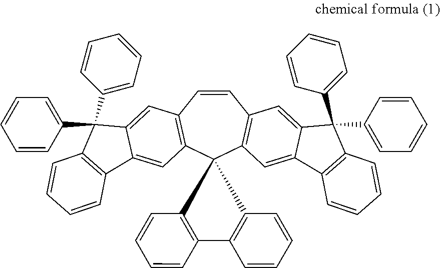

Alternatively, another example is the compound of chemical formula (2), Bis-BnCPSTIF-2, where R′ is represented by General Formula (2), R₁ to R₁₅ and R₁₇ to R₁₈ are all independent hydrogen atoms, and R₁₆ is a methyl group.

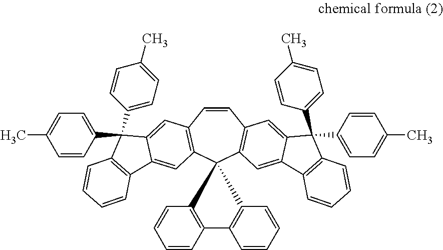

Alternatively, another example is the compound of chemical formula (3), Bis-NPh₂-BnCPSTIF, where R′ is represented by General Formula (2), R₁ to R₆, R₈ to R₁₁, and R₁₃ to R₁₈ are all independent hydrogen atoms, and R₇ and R₁₂ both are diphenyl amino groups.

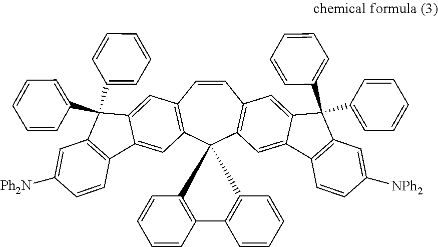

Alternatively, another example is the compound of chemical formula (4a), Bis-CN-BnCPSTIF, where R′ is represented by General Formula (2), R₁ to R₆, R₈ to R₁₁, and R₁₃ to R₁₈ are all independent hydrogen atoms, and R₇ and R₁₂ both are cyano groups.

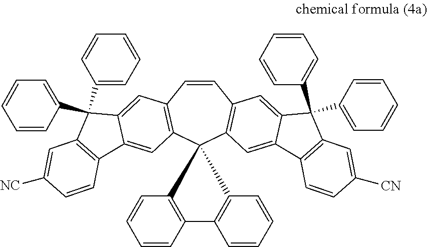

Alternatively, another example is the compound of chemical formula (4b), Bis-Pym-BnCPSTIF, where R′ is represented by General Formula (2), R₁ to R₆, R₈ to R₁₁, and R₁₃ to R₁₈ are all independent hydrogen atoms, and R₇ and R₁₂ both are pyrimidine groups.

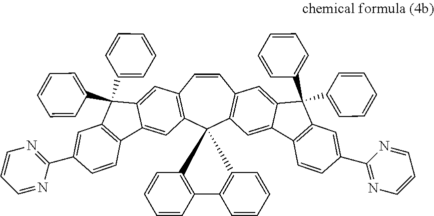

Alternatively, another example is the compound of chemical formula (5), NPh₂-BnCPSTIF-CN, where R′ is represented by General Formula (2), R₁ to R₆, R₈ to R₁₁, and R₁₃ to R₁₈ are all independent hydrogen atoms, R₇ is a cyano group, and R₁₂ is a diphenyl amino group.

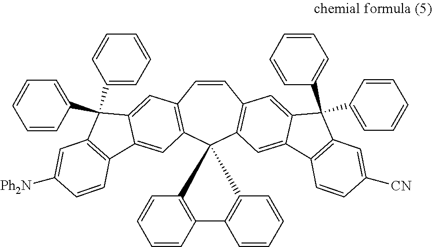

Moreover, in the present embodiment, the spirally configured cis-stilbene/fluorene hybrid materials have glass transition temperatures ranged from 234° C. to 259° C., decomposition temperatures ranged from 469° C. to 492° C., oxidation potentials ranged from 0.33V to 1.03V and reduction potentials ranged from −1.77V to −2.13V. In addition, the highest occupied molecular orbital energy levels (E_{LUMO}) of the spirally configured cis-stilbene/fluorene hybrid materials are ranged from −5.1 eV to −5.8 eV and the lowest unoccupied molecular orbital energy levels (E_{LUMO}) are ranged from −2.7 eV to −3.0 eV.

Organic Electroluminescent Device

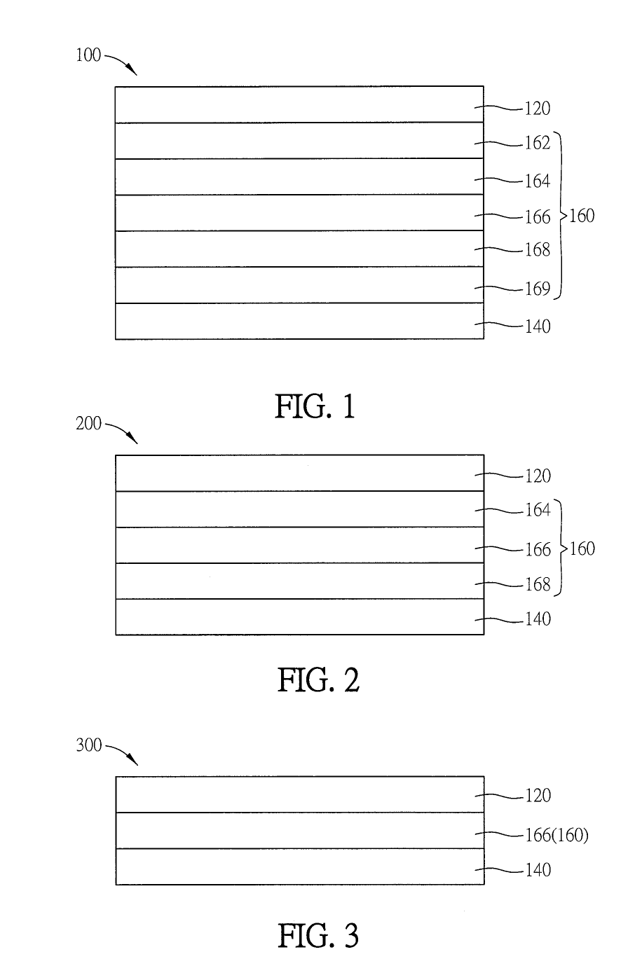

Please refer to FIG. 1, an organic electroluminescent device 100 of the second embodiment according to the invention includes a first electrode layer 120, a second electrode layer 140 and an organic luminescent unit 160. In the embodiment, the first electrode layer 120 can be a transparent electrode material, such as indium tin oxide (ITO), and the second electrode layer 140 can be a metal, transparent conductive substance or any other suitable conductive material, such as aluminum. On the other hand, the first electrode layer 120 can also be a metal, transparent conductive substance or any other suitable conductive material, and the second electrode layer 140 can also be a transparent electrode material. Overall, at least one of the first electrode layer 120 and the second electrode layer 140 of the present embodiment is a transparent electrode material, so that the light emitted from the organic luminescent unit 160 may pass through the transparent electrode, thereby enabling the organic electroluminescent device 100 to emit light.

In addition, please also refer to FIG. 1, the organic luminescent unit 160 can comprise a hole injection layer 162, a hole transport layer 164, an organic luminescent layer 166, an electron transport layer 168 and an electron injection layer 169. The hole transport layer 164, the organic luminescent layer 166 and the electron transport layer 168 are sequentially deposited between the hole injection layer 162 and the electron injection layer 169.

Herein, the materials of the hole injection layer 162 can be poly(3,4-ethylenedioxythiophene) polystyrene sulfonate (PEDOT:PSS), or poly(3,4-ethylenedioxythiophene) (PEDOT). Moreover, the thickness of the hole injection layer 162 of the embodiment is, for example, no more than 40 nm. The materials of the hole transport layer 164 can be 1,1-Bis[4-[N,N′-di(p-tolyl)amino]phenyl]cyclohexane (TAPC), N,N-bis-(1-naphthyl)-N,N-diphenyl-1,1-biphenyl-4,4-diamine (NPB), N—N′-diphenyl-N—N′bis(3-methylphenyl)-[1-1′-biphenyl]-4-4′-diamine (TPD), or 4,4′,4″-tris(9-carbazolyl)triphenylamine (TCTA) and so on. In the embodiment, the hole injection layer 162 and the hole transport layer 164 can increase the injection rate of hole transported from the first electrode layer 120 to the organic luminescent layer 166 and can also reduce the driving voltage of the organic electroluminescent device 100.

In addition, the thickness of the organic luminescent layer 166 can be between 5 nm and 80 nm. The organic luminescent layer 166 may include the host material and the guest material, and the host materials can be the spirally configured cis-stilbene/fluorene hybrid material as shown in General Formula (1).

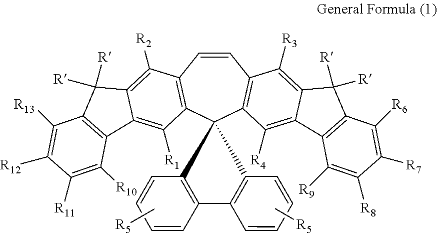

In the General formula (1), R′ is an alkyl group or represented by General Formula (2).

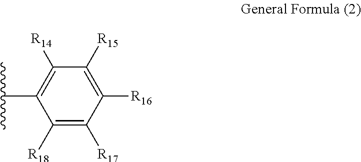

In the General formula (1), R₁ to R₄, R₆, R₈ to R₁₁ and R₁₃ to R₁₈ are each independently selected from the group consisting of a hydrogen atom, halogen atom, cyano group, alkyl group, cycloalkyl group, alkoxy group, amino group, haloalkyl group, thioalkyl group, silyl group and alkenyl group. R₅ is a hydrogen atom, tert-butyl group or aryl group. R₇ and R₁₂ are each independently selected from the group consisting of a hydrogen atom, aryl group, diarylamine, cyano group and hetero aromatic ring (for example, a pyridine or a pyrimidine).

Moreover, the doping concentration (weight percentage) of the guest material according to the embodiment can be ranged from 1 wt % to 20 wt %, for example, 1.5 or 2.0 wt %.

In addition, the guest material can be any suitable material for organic luminescent layer 166, including, but not limited to, Ir(2-phq)₃, Ir(ppy)₃, or FIrpic.

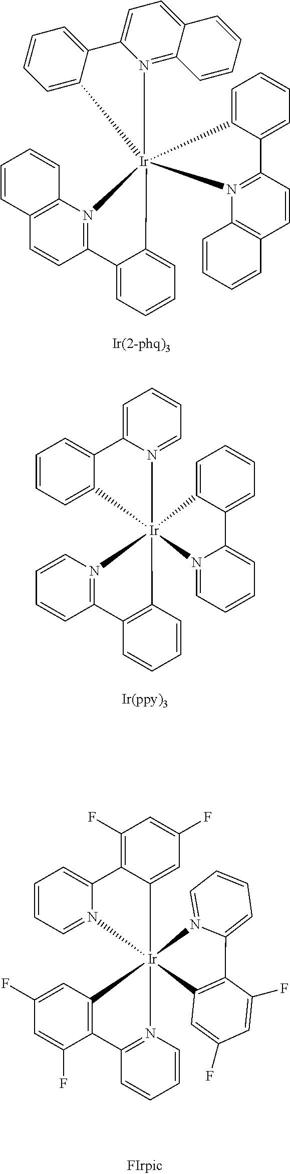

In addition, the spirally configured cis-stilbene/fluorene hybrid material represented by chemical formula (1) can also be a guest material of an organic luminescent layer in an organic electroluminescent device.

The materials of the electron transport layer 168 can be 2,2,2-(1,3,5-benzinetriyl)-tris(1-phenyl-1-H-benzimidazole) (TBPI), or 4,7-diphenyl-1,10-phenanthroline (Bphen). In the embodiment, the thickness of the electron transport layer 168 is, for example, less than 50 nm. The electron transport layer 168 may further increase the transport rate of the electron from the electron injection layer 169 to the organic luminescent layer 166. Moreover, the materials of the electron injection layer 169 can be, for example, LiF. In the embodiment, the thickness of the electron injection layer 169 is, for example, 1.0 nm.

In addition, please refer to FIG. 2, which is a cross-sectional schematic diagram of an organic electroluminescent device 200 of the third embodiment according to the invention. The configuration of the organic electroluminescent device 200 is substantially similar to that of the organic electroluminescent device 100, and same elements have substantially the same characteristics and functions. Therefore, the similar references relate to the similar elements, and detailed explanation is omitted hereinafter.

Please refer to FIG. 2, in the embodiment, the organic luminescent unit 160 can comprise a hole transport layer 164, an organic luminescent layer 166 and an electron transport layer 168. The organic luminescent layer 166 is deposited between the hole transport layer 164 and the electron transport layer 168.

In addition, please refer to FIG. 3, which is a cross-sectional schematic diagram of an organic electroluminescent device 300 of the fourth embodiment according to the invention. The configuration of the organic electroluminescent device 300 is substantially similar with that of the organic electroluminescent device 100, and same elements have substantial the same characteristics and functions. Therefore, the similar references relate to the similar elements, and detailed explanation is omitted hereinafter.

Please refer to FIG. 3, in the embodiment, the organic luminescent unit 160 can comprise an organic luminescent layer 166.

Moreover, the configuration of the organic electroluminescent device according to the invention is not limited to what is disclosed in the second, third or fourth embodiment. The second, third and fourth embodiments are embodiments for illustration.

In addition, the various examples and the selection of the substituents of R′ and R₁ to R₁₈ in General Formula (1) of the spirally configured cis-stilbene/fluorene hybrid materials in the second, third or fourth embodiment, as well as their properties, such as their glass transition temperatures, decomposition temperatures, oxidation potentials, redox potentials, the highest occupied molecular orbital energy levels, and the lowest unoccupied molecular orbital energy levels, are substantially the same as those in the first embodiment and are therefore omitted here.

To illustrate the synthesis of the compounds represented by chemical formula (1) to chemical formula (5), there are several examples shown below.

The synthetic procedure of the compound 1 (chemical formula (1), Bis-BnCPSTIF-1):

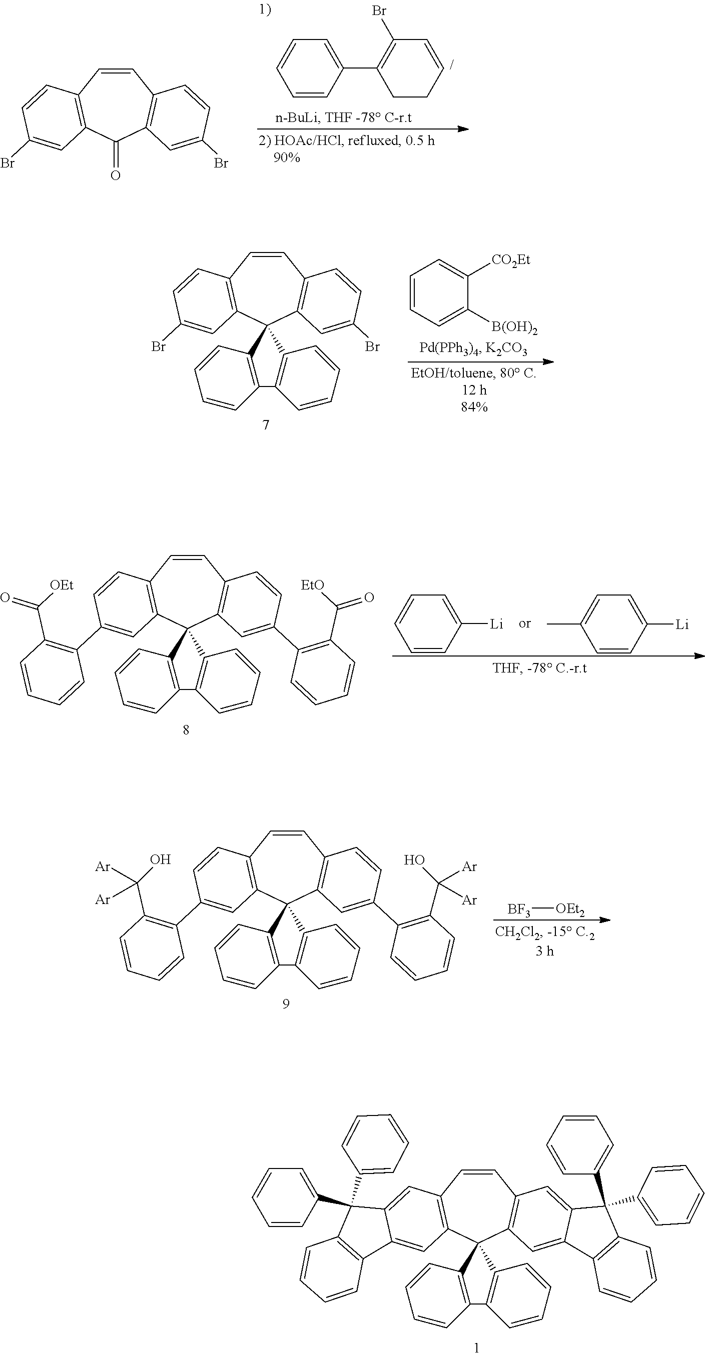

### Example 1: Synthesis of Compound 7 (3,7-dibromo-5,5-spirofluorenyl-5H-dibenzo[a,d]cycloheptene)

A stir bar was placed in a 250 ml two-necked, round-bottomed flask, followed by being dried by vacuum and introduced with nitrogen. An organolithium reagent is then prepared by dropwisely adding 2.5 M n-butyllithium hexane solution (12 ml, 30 mmol) into a solution of 2-bromobiphenyl (6.996 g, 30 mmol) dissolved in anhydryous tetrahydrofuran (100 ml) and placed in the 250 ml flask at −78° C., followed by reaction for 30 minutes. 3,7-dibromo-dibenzo[a,d]cyclo-hepten-5-one (7.28 g, 20 mmol) was dissolved in anhydrous tetrahydrofuran (60 ml) and placed into a 500 ml two-necked flask after the 500 ml flask was dried by vacuum and introduced with nitrogen. The prepared organolilthium reagent was then drop-wisely added into the 500 ml flask containing the dissolved 3,7-dibromo-dibenzo[a,d]cyclo-hepten-5-one through a double-tipped needle. After the temperature of the reaction mixture was returned to the room temperature, 10 ml of saturated aqueous sodium carbonate was added into the reaction mixture to quench the reaction. The reaction mixture was then extracted with dichloromethane (3×100 ml). The combined extractant was then sequentially treated by adding magnesium sulfate (about 5 g) to dry, filtrating, and removing solvent by rotator evaporation, so as to obtain an intermediate product.

The intermediate product dissolved in 30 ml of acetic acid was placed in a 100 ml single-necked, round-bottomed flask with another stir bar. After added with 1 ml of concentrated (12N) hydrogen chloride, the resultant solution turned into a purple-red color. The 100 ml flask containing the reaction mixture was then placed in an 120° C. oil bath and installed with a reflux apparatus. After refluxing for 30 minutes, the flask was then lifted up from the oil bath for cooling down to the room temperature. The flask was then removed from the reflux apparatus and further cooled down to 0° C., followed by adding 40 ml of n-hexane. The reaction mixture was then filtered through a suction funnel to collect solid precipitates, followed by washing with n-hexane for three times. The solid precipitates were recrystallized with a dichoromethane/n-hexane solution to obtain the compound 7 as colorless crystals (9.011 g, yield: 90%).

Spectral data as follow: Tₘ 283° C. (DSC); M.W.: 500.22; ¹H NMR (400 MHz, CDCl₃) δ 7.91 (d, J=7.7 Hz, 2H), 7.77 (d, J=7.6 Hz, 2H), 7.43 (t, J=7.5 Hz, 2H), 7.33 (dd, J=8.2, 2.0 Hz, 2H), 7.30 (d, J=7.6 Hz, 2H), 7.21 (d, J=8.2 Hz, 2H), 6.99 (d, J=1.9 Hz, 2H), 6.90 (s, 2H); ¹³C NMR (100 MHz, CDCl₃) δ 151.5, 143.4, 138.8, 135.2, 133.5, 132.6, 131.9, 130.5, 128.6, 127.9, 126.6, 122.8, 120.6, 65.2; MS (FAB) 500.0 (M⁺, 28); TLC R_{f} 0.35 (CH₂Cl₂/hexanes, 1/5); High Resolution-MS calcd for C₂₇H₁₆Br₂: 499.9598, found: 499.9600.

### Example 2: Synthesis of Compound 8 (diethyl 2,2′-(spiro[dibenzo[a,d][7]annulene-5,9′-fluorene]-3,7-diyl)di-benzoate)

A stir bar was placed in a 50 ml two-necked round-bottomed flask, followed by being dried by vacuum and introduced with nitrogen. The compound 7 (501 mg, 1 mmol), potassium carbonate (690 mg, 5 mmol), Pd(PPh₃)₄ as catalyst, 8 ml of toluene and 2 ml of ethanol were added into the 50 ml flask, followed by stirring for 10 minutes. After stirring, 2-(ethoxycarbonyl) phenylboronic acid (580 mg, 2.1 mmol) was added. The resulting mixture was then heated to 80° C. After reacting for 24 hours, the reaction mixture was then cooled down to the room temperature, followed by quenching with 20 ml water and then being extracted with dichloromethane (3×20 ml). The combined organic extractant was added with magnesium sulfate (about 500 mg) to dry, followed by filtration and condensation with a rotary evaporator to remove the solvent, so as to obtain a crude product. The crude product was then purified through column chromatography using a mixture of dichloromethane and n-hexane (2:1) as eluent and compound 8 (537 mg, yield: 84%) as a white solid was then obtained.

Spectral data as follow: Tₘ 138° C. (DSC); M.W.: 638.74; ¹H NMR (400 MHz, CDCl₃) δ 8.00 (d, J=8.0 Hz, 2H), 7.70 (d, J=7.6 Hz, 2H), 7.67 (d, J=8.0 Hz, 2H), 7.42 (d, J=7.6 Hz, 2H), 7.37 (t, J=7.6 Hz, 2H), 7.32 (t, J=7.6 Hz, 2H), 7.28 (t, J=8.4 Hz, 2H), 7.19 (d, J=8.0 Hz, 2H), 7.19 (t, J=8.0 Hz, 2H), 7.11 (d, J=7.6 Hz, 2H), 7.04 (s, 2H), 6.89 (s, 2H), 3.71 (q, J=7.2 Hz, 4H), 0.63 (t, J=7.2 Hz, 6H); ¹³C NMR (100 MHz, CDCl₃) δ 168.4, 152.3, 141.8, 141.4, 138.8, 135.4, 133.0, 132.0, 131.0, 130.9, 130.2, 129.7, 129.1, 128.0, 127.4, 127.1, 126.95, 126.90, 120.2, 65.9, 60.7, 13.2; TLC R_{f} 0.30 (CH₂Cl₂/hexanes, 2/1); High Resolution-MS calcd for C₄₅H₃₄O₆: 638.2457, found: 638.2450.

### Example 3: Synthesis of Compound 1 (12,12,17,17-tetraphenyl-12,17-dihydrospiro[cyclohepta[1,2-b:5,4-b′]-difluorene-6,9′-fluorene])

A stir bar was placed in a 50 ml two-necked round-bottomed flask, followed by being dried by vacuum and introduced with nitrogen. A reaction mixture was prepared by dropwisely adding 2.0 M phenyllithium di-n-butyl ether solution (2.75 ml, 5.5 mmol) into a solution of the compound 8 (639 mg, 1 mmol) dissolved in anhydryous tetrahydrofuran (100 ml) and placed in the 50 ml flask at −78° C., followed by reaction for 3 hours. After the temperature of the reaction mixture was returned to the room temperature, 3 ml of saturated aqueous sodium bicarbonate solution was introduced to quench the reaction. The resulting mixture was then extracted with dichloromethane (3×20 ml). The obtained organic extractant was added with magnesium sulfate (about 500 mg) to dry, followed by filtration and condensation with a rotary evaporator to remove the solvent, so as to obtain an intermediate product 9 which is a tertiary alcohol.

The intermediate product 9 was dissolved in 100 ml of anhydrous dichloromethane and placed in another 250 ml flask with another stir bar. The flask containing the reaction mixture was placed on a salt-ice bath to cool down to −15° C., followed by adding with 100 μl of boron trifluoride diethyl etherate (BF₃—OEt₂, conc. 48 wt %, 9.7 M, 0.97 mmol). The color of the resulting solution then became deep-purple. After reaction for 3 hours, 3 ml of deionized water was added to quench the reaction, followed by condensation with a rotary evaporator to remove the solvent and then being extracted by dichloromethane (3×20 ml). The obtained organic extractant was added with magnesium sulfate (about 500 mg) to dry, followed by filtration and condensation with a rotary evaporator to remove the solvent, so as to obtain a crude product. The crude product was purified by column chromatography using a mixture of dichloromethane and n-hexane (1:2) as eluent and compound 1 (585 mg, yield: 71%) as a white solid was then obtained.

Spectral data as follow: Tₘ 465° C. (DSC); M.W.: 823.02; ¹H NMR (400 MHz, CDCl₃) δ 8.10 (d, J=7.6 Hz, 2H), 7.84 (d, J=7.6 Hz, 2H), 7.48 (t, J=7.6 Hz, 2H), 7.36 (d, J=2.0 Hz, 4H), 7.32 (t, J=8.0 Hz, 4H), 7.26-7.15 (m, 26H), 6.89 (s, 2H); ¹³C NMR (100 MHz, CDCl₃) δ 152.8, 151.3, 149.8, 145.6, 141.3, 140.3, 139.7, 139.1, 136.3, 133.2, 129.3, 128.2, 128.1, 127.7, 127.6, 127.4, 127.3, 126.6, 126.1, 120.7, 120.4, 120.0, 66.2, 65.0; TLC R_{f} 0.50 (CH₂Cl₂/hexanes, 1/2); High Resolution-MS calcd for C₆₅H₄₂: 822.3287, found: 822.3284.

The synthetic procedure of the compound 2 (chemical formula (2), Bis-BnCPSTIF-2):

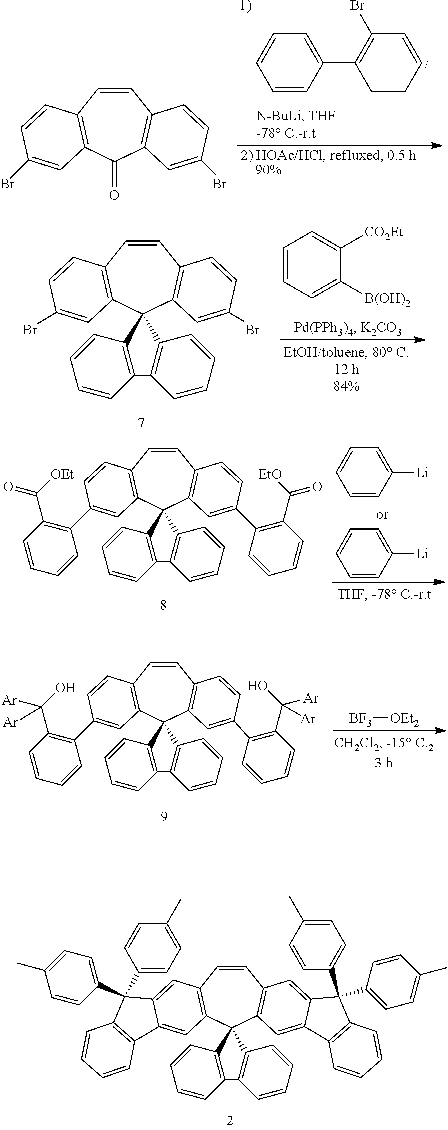

### Example 4: Synthesis of Compound 2 (12,12,17,17-tetra-p-tolyl-12,17-dihydrospiro[cyclohepta[1,2-b:5,4-b]difluorene-6,9′-fluorene])

A stir bar was placed in a 250 ml two-necked round-bottomed flask, followed by being dried by vacuum and introducing with nitrogen. An organolithium reagent is then prepared by dropwisely adding 2.5 M n-butyllithium hexane solution (5.6 ml, 14 mmol) into a solution of 2-bromobiphenyl (2.395 g, 14 mmol) dissolved in anhydryous tetrahydrofuran (150 ml) and placed in the 250 ml flask at −78° C., followed by reaction for 30 minutes. The compound 8 (1.278 g, 2 mmol) was dissolved in anhydrous tetrahydrofuran (50 ml) and placed into a 500 ml double-neck flask after the 500 ml flask was dried by vacuum and introduced with nitrogen. The prepared organolilthium reagent was then drop-wisely added into the 500 ml flask containing the dissolved compound 8 through a double-tipped needle. After the temperature of the reaction mixture was returned to the room temperature, 10 ml of saturated aqueous sodium carbonate was added into the reaction mixture to quench the reaction. The reaction mixture was then extracted with dichloromethane (3×50 ml). The combined extractant was then sequentially treated by adding magnesium sulfate (about 1 g) to dry, filtrating, and removing solvent by rotator evaporation, so as to obtain an intermediate product 9 which is a tertiary alcohol.

The intermediate product 9 was dissolved in 200 ml of anhydrous dichloromethane and placed in another 500 ml flask with another stir bar. The flask containing the reaction mixture was placed on a salt-ice bath to cool down to −15° C., followed by adding with 200 μl of boron trifluoride diethyl etherate (BF₃—OEt₂, conc. 48 wt %, 9.7 M, 1.94 mmol). The color of the resulting solution then became deep-purple. After reaction for 3 hours, 6 ml of deionized water was added to quench the reaction, followed by condensation with a rotary evaporator to remove the solvent and then being extracted by dichloromethane (3×50 ml). The obtained organic extractant was added with magnesium sulfate (about 1 g) to dry, followed by filtration and condensation with a rotary evaporator to remove the solvent, so as to obtain a crude product. The crude product was purified by column chromatography using a mixture of dichloromethane and n-hexane (1:3) as eluent and compound 2 (999 mg, yield: 61%) as a white solid was then obtained.

Spectral data as follow: Tₘ 417° C. (DSC); M.W.: 879.13; ¹H NMR (400 MHz, CDCl₃) δ 8.09 (d, J=8.0 Hz, 2H), 7.83 (d, J=7.6 Hz, 2H), 7.42 (t, J=7.6 Hz, 2H), 7.34 (s, 4H), 7.33-7.28 (m, 4H), 7.25-7.14 (m, 6H), 7.02 (q, J=8.0 Hz, 16H), 6.88 (s, 2H), 2.27 (s, 12H); ¹³C NMR (100 MHz, CDCl₃) δ 152.8, 151.7, 150.2, 142.8, 141.2, 140.2, 139.6, 139.2, 136.3, 136.1, 133.2, 129.2, 128.8, 128.1, 128.0, 127.7, 127.6, 127.3, 127.2, 126.0, 120.6, 120.3, 119.9, 66.3, 64.4, 20.9; TLC R_{f} 0.40 (CH₂Cl₂/hexanes, 1/3); High Resolution-MS calcd for C₆₉H₅₀: 878.3913, found: 878.3928.

The synthetic procedure of the compound 3 (chemical formula (3), Bis-NPh₂-BnCPSTIF):

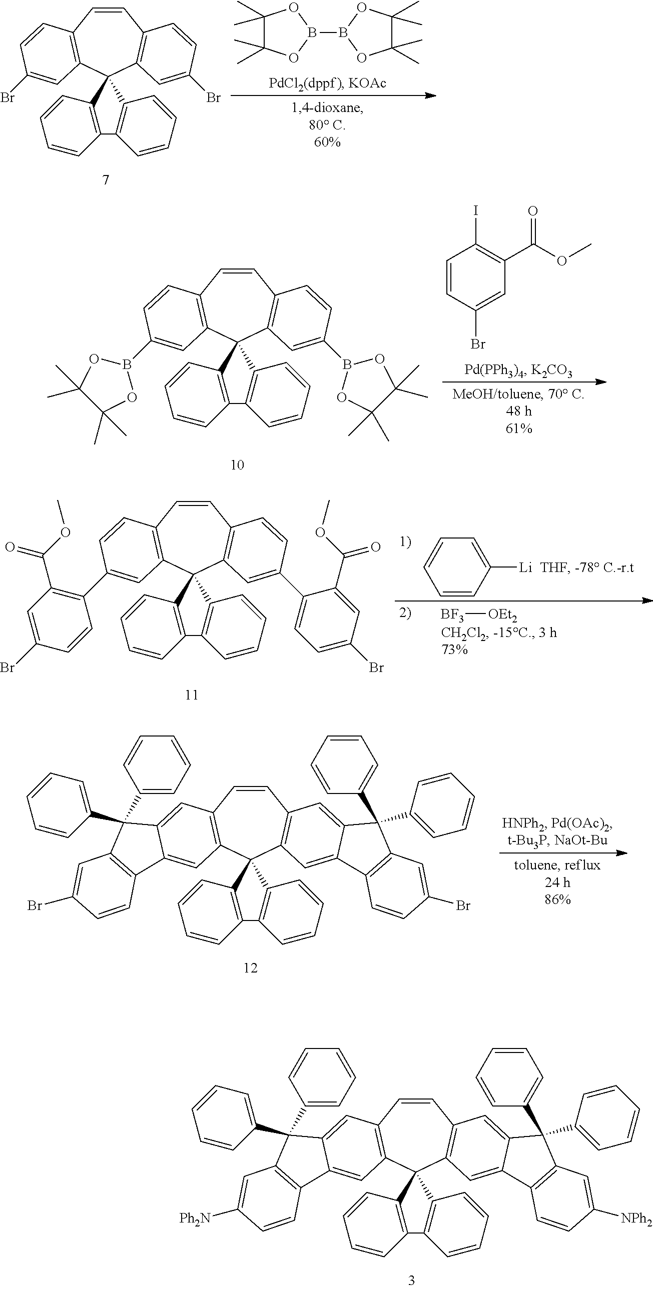

### Example 5: Synthesis of Compound 10 (3,7-bis(4,4,5,5-tetramethyl-1,3,2-dioxaborolan-2-yl)spiro[dibenzo[a,d][7]annulene-5,9′-fluorene])

A stir bar was placed in a 50 ml two-necked rounded-bottom flask, followed by being dried by vacuum and introduced with nitrogen. The compound 7 (501 mg, 1 mmol), potassium acetate (410 mg, 5 mmol), the catalyst PdCl₂(dppf) (40 mg, 0.05 mmol), bis(pinacolato)diboron (635 mg, 2.5 mmol) and 1,4-dioxane (10 ml, removed oxygen in advance by introducing with argon) were added in the 50 ml flask. The reaction mixture was then stirred at 80° C. for 24 hours. After being cooled down to the room temperature, the solvent was removed by a rotary evaporator and then extracted by dichloromethane (3×20 ml). The obtained organic extractant was added with magnesium sulfate (about 1 g) to dry, followed by filtration and condensation with a rotary evaporator to remove the solvent, so as to obtain a crude product. The crude product was purified by column chromatography using a mixture of acetone and n-hexane (1:4) as eluent and compound 10 (357 mg, yield: 60%) as a white solid was then obtained.

Spectral data as follow: Tₘ 207° C. (DSC); M.W.: 594.35; ¹H NMR (400 MHz, CDCl₃) δ 7.95 (d, J=8.0 Hz, 2H), 7.73 (d, J=7.6 Hz, 2H), 7.61 (d, J=7.2 Hz, 2H), 7.40 (s, 2H), 7.35 (t, J=6.8 Hz, 4H), 7.24 (t, J=8.0 Hz, 2H), 7.01 (s, 2H), 1.20 (s, 24H); ¹³C NMR (100 MHz, CDCl₃) δ 152.4, 141.2, 139.0, 138.8, 135.3, 134.3, 133.3, 131.4, 127.7, 127.1, 127.0, 120.2, 83.4, 66.0, 24.7; MS (EI, 20 eV) 594.3 (M⁺, 68); TLC R_{f} 0.35 (acetone/hexanes, 1/1); High Resolution-MS calcd for C₃₉H₄₀B₂O₄: 594.3113, found: 594.3120.

### Example 6: Synthesis of Compound 11 (Dimethyl 6,6′-(spiro[dibenzo[a,d][7]annulene-5,9′-fluorene]-3,7-diyl)bis(3-bromobenzoate))

A stir bar was placed in a 100 ml two-necked rounded-bottom flask. The 100 ml flask was then installed with a reflux system, followed by being dried by vacuum and introduced with nitrogen. The compound 10 (1486 mg, 0.05 mmol), potassium carbonate (1210 mg, 8.75 mmol), the catalyst Pd(PPh₃)₄ (144 mg, 0.05 mmol), toluene (22.5 ml), methanol (7.5 ml) and 2-iodo-5-bromo-benzoic acid methyl ester (1761 mg, 5.75 mmol) were added in the 100 ml flask. The reaction mixture was then stirred at 70° C. for 48 hours. After being cooled down to the room temperature, the reaction mixture was introduced with oxygen to quench the reaction and then extracted by dichloromethane (3×20 ml). The obtained organic extractant was added with magnesium sulfate (about 1 g) to dry, followed by filtration and condensation with a rotary evaporator to remove the solvent, so as to obtain a crude product. The crude product was purified by column chromatography using a mixture of dichloromethane and n-hexane (3:1) as eluent and compound 11 (1172 mg, yield: 61%) as a white solid was then obtained.

Spectral data as follow: Tₘ 138° C. (DSC); M.W.: 768.49; H NMR (400 MHz, CDCl₃) δ 7.94 (d, J=8.0 Hz, 2H), 7.79 (d, J=2.0 Hz, 2H), 7.74 (d, J=7.6 Hz, 2H), 7.49 (dd, J=8.0, 2.0 Hz, 2H), 7.41 (d, J=7.6 Hz, 2H), 7.33 (t, J=7.6 Hz, 2H), 7.18 (t, J=7.6 Hz, 2H), 7.16 (dd, J=8.0, 1.2 Hz, 2H), 7.03 (s, 2H), 6.96 (d, J=8.4 Hz, 2H), 6.79 (d, J=1.6 Hz, 2H), 3.33 (s, 6H); ¹³C NMR (100 MHz, CDCl₃) δ 167.3, 152.3, 141.5, 140.7, 139.9, 138.8, 135.8, 134.1, 133.2, 132.7, 132.3, 132.1, 131.9, 129.1, 128.2, 127.5, 126.92, 126.87, 121.0, 120.3, 65.9, 51.9; TLC R_{f} 0.35 (CH₂C12/hexanes, 3/1); High Resolution-MS calcd for C₄₃H₂₈Br₂O₄: 766.0354, found: 766.0347.

### Example 7: Synthesis of Compound 12 (2,10-dibromo-12,12,17,17-tetraphenyl-12,17-dihydrospiro[cyclohepta[1,2-b:5,4-b′]difluorene-6,9′-fluorene])

A stir bar was placed in a 250 ml two-necked round-bottomed flask, followed by being dried by vacuum and introduced with nitrogen. A reaction mixture was prepared by dropwisely adding 2.0 M phenyllithium di-n-butyl ether solution (7.7 ml, 15.4 mmol) into a solution of the compound 11 (2.152 mg, 2.8 mmol) dissolved in anhydryous tetrahydrofuran (60 ml) and placed in the 250 ml flask at −78° C., followed by reaction for 3 hours. After the temperature of the reaction mixture was returned to the room temperature, 15 ml of saturated aqueous sodium bicarbonate solution was introduced to quench the reaction. The resulting mixture was then extracted with dichloromethane (3×50 ml). The obtained organic extractant was added with magnesium sulfate (about 1.5 g) to dry, followed by filtration and condensation with a rotary evaporator to remove the solvent, so as to obtain an intermediate product which is a tertiary alcohol.

The intermediate product was dissolved in 280 ml of anhydrous dichloromethane and placed in a 500 ml single-neck rounded-bottom flask with another stir bar. The flask containing the reaction mixture was placed on a salt-ice bath to cool down to −15° C., followed by adding with 280 μl of boron trifluoride diethyl etherate (BF₃—OEt₂, conc. 48 wt %, 9.7 M, 2.72 mmol). The color of the resulting solution then became deep-purple. After reaction for 3 hours, 10 ml of deionized water was added to quench the reaction, followed by condensation with a rotary evaporator to remove the solvent and then being extracted by dichloromethane (3×50 ml). The obtained organic extractant was added with magnesium sulfate (about 1.5 g) to dry, followed by filtration and condensation with a rotary evaporator to remove the solvent, so as to obtain a crude product. The crude product was purified by column chromatography using a mixture of dichloromethane and n-hexane (1:2) as eluent and compound 12 (1950 mg, yield: 73%) as a white solid was then obtained.

Spectral data as follow: Tₘ 446° C. (DSC); M.W.: 980.82; ¹H NMR (400 MHz, CDCl₃) δ 8.06 (d, J=8.6 Hz, 2H), 7.83 (d, J=7.6 Hz, 2H), 7.47 (t, J=7.6 Hz, 2H), 7.41 (d, J=1.6 Hz, 2H), 7.36-7.29 (m, 8H), 7.22-7.18 (m, 12H), 7.13-7.11 (m, 8H), 7.08 (d, J=8.4 Hz, 2H), 6.88 (s, 2H); ¹³C NMR (100 MHz, CDCl₃) δ 153.3, 152.5, 149.7, 144.9, 141.5, 139.2, 139.1, 138.7, 136.6, 133.3, 130.6, 129.35, 129.32, 128.4, 128.3, 128.0, 127.50, 127.47, 126.9, 121.4, 121.3, 120.8, 120.5, 66.2, 65.1; TLC R_{f} 0.30 (CH₂Cl₂/hexanes, 1/2); High Resolution-MS calcd for C₆₅H₄₀Br₂: 978.1497, found: 978.1486.

### Example 8: Synthesis of Compound 3 (N²,N²,N¹⁰,N¹⁰,12,12,17,17-octaphenyl-12,17-dihydrospiro[cyclohepta[1,2-b:5,4-b′]difluorene-6,9′-fluorene]-2,10-diamine)

A stir bar was placed in a 25 ml two-necked rounded-bottom flask. The 25 ml flask was then installed with a reflux system, followed by being dried by vacuum and introduced with nitrogen. The compound 12 (491 mg, 0.5 mmol), the catalyst Pd(OAc)₂ (6 mg, 0.025 mmol), sodium tert-butoxide (NaOt-Bu, 116 mg, 1.2 ml), and dipheyl amine (186 mg, 1.1 mmol) were placed in the 25 ml flask and dissolved in 10 ml of anhydrous toluene, followed by adding with a toluene solution of 0.04 M tri(tert-butyl)phosphine (t-Bu₃P, 2.5 ml, 0.1 mmol). The reaction mixture was then refluxed for 24 hours. After being cooled down to the room temperature, the reaction mixture was added with saturated aqueous sodium bicarbonate (5 ml) solution to quench the reaction and then extracted by trichloromethane (3×20 ml). The obtained organic extractant was added with magnesium sulfate (about 500 mg) to dry, followed by filtration and condensation with a rotary evaporator to remove the solvent, so as to obtain a crude product. The crude product was purified by column chromatography using a mixture of trichloromethane and n-hexane (1:2) as eluent and compound 3 (498 mg, yield: 86%) as a yellow solid was then obtained.

Spectral data as follow: Tₘ 360° C. (DSC); M.W.: 1157.44; ¹H NMR (400 MHz, CDCl₃) δ 8.04 (d, J=8.0 Hz, 2H), 7.78 (d, J=7.6 Hz, 2H), 7.41 (t, J=7.2 Hz, 2H), 7.28 (s, 4H), 7.21 (s, 2H), 7.17-7.05 (m, 32H), 6.98-6.83 (m, 16H); ¹³C NMR (100 MHz, CDCl₃) δ 153.0, 152.7, 149.4, 147.50, 147.45, 145.5, 141.3, 140.0, 139.1, 135.5, 134.1, 132.9, 129.4, 129.1, 128.1, 128.09, 128.04, 127.5, 127.3, 126.5, 124.1, 122.9, 122.8, 121.3, 120.5, 120.3, 120.1, 66.3, 65.0; TLC R_{f} 0.35 (CHCl₃/hexanes, 1/2); High Resolution-MS calcd for C₈₉H₆₀N₂: 1156.4756, found: 1156.4764.

The synthetic procedure of the compound 4a (chemical formula (4a), Bis-CN-BnCPSTIF):

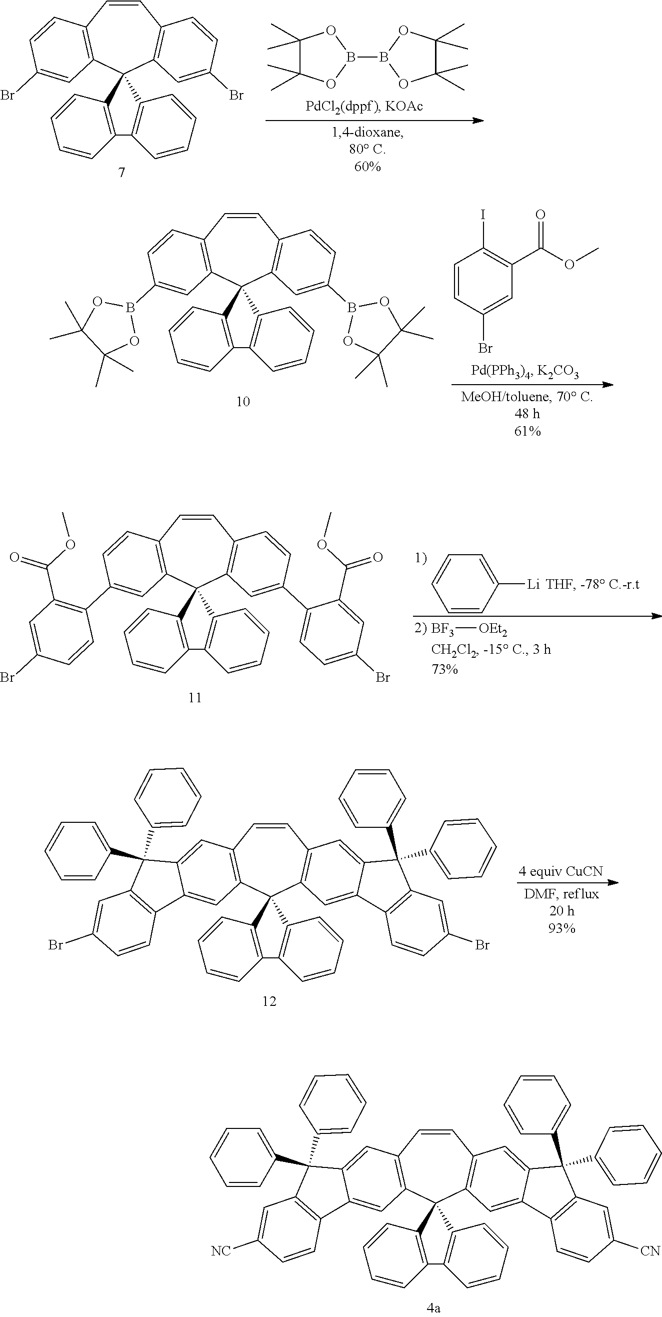

### Example 9: Synthesis of Compound 4a (12,12,17,17-tetraphenyl-12,17-dihydrospiro[cyclohepta[1,2-b:5,4-b′]difluorene-6,9′-fluorene]-2,10-dicarbonitrile)

A stir bar was placed in a 10 ml two-necked rounded-bottom flask. The 10 ml flask was then installed with a reflux system, followed by being dried by vacuum and introduced with nitrogen. The compound 12 (491 mg, 0.5 mmol), copper(I) cyanide (180 mg, 2 mmol), and 2.5 ml of N,N-dimethyformide (DMF) were added in the 10 ml flask. The reaction mixture was refluxed for 20 hours and then was cooled down to the room temperature. Distilled water (20 ml), FeCl₃ (3.5 g, 22 mmol), and 2 ml concentrated (12N) hydrogen chloride aqueous solution were added into a 50 ml rounded-bottom flask and well mixed. The reaction mixture in the 10 ml flask was then added into the 50 ml flask, followed by heating to 70° C. for 30 minutes. The resulting mixture was then extracted by trichloromethane (3×20 ml). The obtained organic extractant was added with magnesium sulfate (about 300 mg) to dry, followed by filtration and condensation with a rotary evaporator to remove the solvent, so as to obtain a crude product. The crude product was purified by column chromatography using a mixture of dichloromethane and n-hexane (1:1) as eluent and compound 4a (406 mg, yield: 86%) as a white solid was then obtained.

Spectral data as follow: Tₘ 429° C. (DSC); M.W.: 873.04; ¹H NMR (400 MHz, CDCl₃) δ 8.04 (d, J=7.6 Hz, 2H), 7.85 (d, J=7.6 Hz, 2H), 7.57 (s, 2H), 7.50 (t, J=6.4 Hz, 2H), 7.50 (d, J=9.2 Hz, 2H), 7.38 (s, 2H), 7.37 (s, 2H), 7.35 (t, J=7.2, 1.2 Hz, 2H), 7.28 (t, J=8.0 Hz, 2H), 7.24-7.19 (m, 12H), 7.11-7.08 (m, 8H), 6.94 (s, 2H); ¹³C NMR (100 MHz, CDCl₃) δ 152.2, 152.1, 150.7, 144.2, 144.1, 141.9, 139.1, 138.4, 137.8, 133.8, 131.7, 129.8, 129.5, 128.5, 127.9, 127.6, 127.3, 127.2, 121.8, 120.6, 119.3, 110.6, 66.1, 65.2; TLC R_{f} 0.25 (CH₂Cl₂/hexanes, 1/1); High Resolution-MS calcd for C₆₇H₄₀N₂: 872.3139, found: 872.3189.

The synthetic procedure of the compound 4b (chemical formula (4b), Bis-Pym-BnCPSTIF):

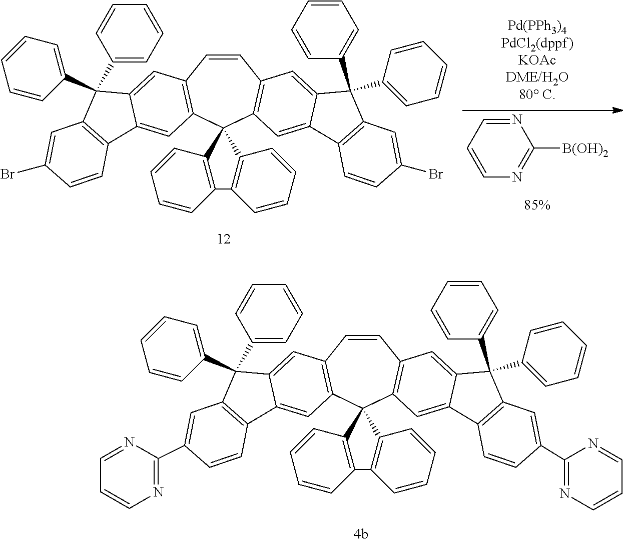

### Example 10: Synthesis of Compound 4b (12,12,17,17-tetraphenyl-12,17-dihydrospiro[cyclohepta[1,2-b:5,4-b′]difluorene-6,9′-fluorene]-2,10-di-1,6-pyrimidine)

A stir bar was placed in a 250 ml two-necked rounded-bottom flask. The 250 ml flask was then installed with a reflux system, followed by being dried by vacuum and introduced with nitrogen. The compound 12 (2945 mg, 3 mmol), 2-pyrimidinylboronic acid (893 mg, 7.2 mmol), potassium carbonate (1656 mg, 12 mmol), and the catalyst Pd(PPh₃)₄ (172 mg, 0.15 mmol) were sequentially added into the 250 flask, followed by adding with 1,2-dimethoxyethane (DME, 100 ml, deoxygenated in advance) and distilled water (20 ml). The reaction mixture was refluxed for 48 hours. After being cooled down to the room temperature, the reaction mixture was condensed by a rotary evaporator to remove the solvent and extracted by dichloromethane (3×150 ml). The obtained organic extractant was then added with magnesium sulfate (about 5.0 g) to dry, followed by filtration and condensation with a rotary evaporator to remove the solvent, so as to obtain a crude product. The crude product was purified by column chromatography using a mixture of acetone and n-hexane (1:2) as eluent and recrystallized to obtain compound 4b (2430 mg, yield: 85%) as a mild yellow solid.

Spectral data as follow: Tₘ 442° C. (DSC); M.W.: 979.20; ¹H NMR (400 MHz, CDCl₃) δ 8.68 (d, J=7.6, 4H), 8.06 (d, J=7.8 Hz, 2H), 7.88 (d, J=7.6 Hz, 2H), 7.77 (t, J=6.4, 2H), 7.66 (s, 2H), 7.47 (dd, J=8.2, 1.8 Hz, 2H), 7.40 (s, 2H), 7.38 (s, 2H), 7.33 (t, J=7.2 Hz, 2H), 7.28 (t, J=8.0 Hz, 2H), 7.18-7.13 (m, 14H), 7.12-7.10 (m, 8H), 6.92 (s, 2H); ¹³C NMR (100 MHz, CDCl₃) δ 167.5, 158.3, 152.5, 148.1, 150.69, 144.3, 141.6, 140.0, 139.5, 138.9, 138.6, 137.6, 131.3, 131.9, 129.1, 128.7, 128.7, 128.5, 128.1, 126.8, 125.1, 122.2, 120.4, 116.3, 66.9, 65.6; TLC R_{f} 0.28 (acetone/hexanes, 1/2); High Resolution-MS calcd for C₇₃H₄₆N₄: 978.3722, found: 978.3689.

The synthetic procedure of the compound 5 (chemical formula (5), NPh₂-BnCPSTIF-CN):

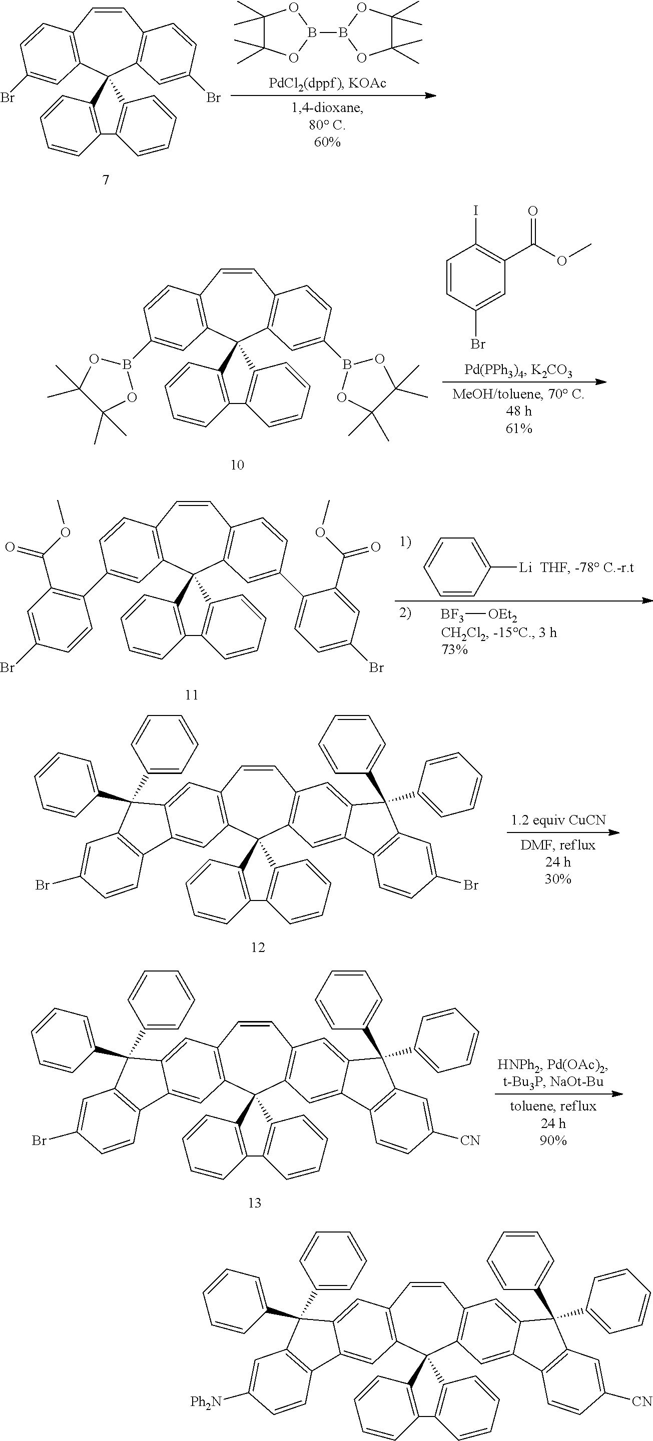

### Example 11: Synthesis of Compound 13 (2-bromo-12,12,17,17-tetraphenyl-12,17-dihydrospiro[cyclohepta[1,2-b:5,4-b′]difluorene-6,9′-fluorene]-10-carbonitrile)

A stir bar was placed in a 10 ml two-necked rounded-bottom flask. The 10 ml flask was then installed with a reflux system, followed by being dried by vacuum and introduced with nitrogen. The compound 12 (491 mg, 0.5 mmol), copper(I) cyanide (54 mg, 0.6 mmol), and 2.5 ml of N,N-dimethyformide were added in the 10 ml flask. The reaction mixture was refluxed for 20 hours and then was cooled down to the room temperature. Distilled water (20 ml), FeCl₃ (3.5 g, 22 mmol), and 2 ml of concentrated (12N) hydrogen chloride aqueous solution were added into a 50 ml rounded-bottom flask and well mixed. The reaction mixture in the 10 ml flask was then added into the 50 ml flask, followed by heating to 70° C. for 30 minutes. The resulting mixture was then extracted by trichloromethane (3×20 ml). The obtained organic extractant was added with magnesium sulfate (about 300 mg) to dry, followed by filtration and condensation with a rotary evaporator to remove the solvent, so as to obtain a crude product. The crude product was purified by column chromatography using a mixture of dichloromethane and n-hexane (1:2) as eluent and compound 13 (139 mg, yield: 30%) as a yellow solid was then obtained.

Spectral data as follow: Tₘ 420° C. (DSC); M.W.: 926.93; ¹H NMR (400 MHz, CDCl₃) δ 8.07 (d, J=8.0 Hz, 2H), 7.85 (d, J=8.0 Hz, 2H), 7.58-7.43 (m, 6H), 7.39-3.28 (m, 8H), 7.23-7.20 (m, 12H), 7.14-7.09 (m, 8H), 6.92 (q, J=12.4 Hz, 2H); ¹³C NMR (100 MHz, CDCl₃) δ 153.33, 153.32, 152.4, 152.0, 150.6, 149.81, 149.80, 144.79, 144.78, 144.22, 144.21, 141.92, 141.91, 141.5, 139.43, 139.42, 139.12, 139.10, 138.60, 138.59, 138.14, 138.13, 138.03, 138.02, 136.43, 136.42, 134.2, 133.0, 131.6, 130.6, 129.8, 129.5, 129.35, 129.34, 128.5, 128.44, 128.38, 128.0, 127.87, 127.86, 127.5, 127.4, 127.2, 126.9, 121.7, 121.6, 121.5, 121.4, 120.8, 120.5, 119.3, 110.48, 110.47, 66.1, 65.2; TLC R_{f} 0.35 (CH₂Cl₂/hexanes, 1/2); High Resolution-MS calcd for C₆₆H₄₀BrN: 925.2344, found: 925.2351.

### Example 12: Synthesis of Compound 5 (10-(diphenylamino)-12,12,17,17-tetraphenyl-12,17-dihydrospiro[cyclohepta[1,2-b:5,4-b′ ]difluorene-6,9′-fluorene]-2-carbonitrile)

A stir bar was placed in a 50 ml two-necked rounded-bottom flask. The 50 ml flask was then installed with a reflux system, followed by being dried by vacuum and introduced with nitrogen. The compound 13 (377 mg, 0.4 mmol), the catalyst Pd(OAc)₂ (5 mg, 0.022 mmol), sodium tert-butoxide (137 mg, 1.43 ml), and dipheylamine (206 mg, 1.22 mmol) were placed in the 50 ml flask and dissolved in 20 ml of anhydrous toluene, followed by adding with a toluene solution of 0.04 M tri(tert-butyl)phosphine (2.5 ml, 0.1 mmol). The reaction mixture was then refluxed for 24 hours. After being cooled down to the room temperature, the reaction mixture was added with saturated aqueous sodium bicarbonate (5 ml) solution to quench the reaction and then extracted by trichloromethane (3×20 ml). The obtained organic extractant was added with magnesium sulfate (about 200 mg) to dry, followed by filtration and condensation with a rotary evaporator to remove the solvent, so as to obtain a crude product. The crude product was purified by column chromatography using a mixture of dichloromethane and n-hexane (1:1) as eluent and compound 5 (370 mg, yield: 90%) as a yellow solid was then obtained.

Spectral data as follow: Tₘ 349° C. (DSC); M.W.: 1015.24; ¹H NMR (400 MHz, CDCl₃) δ 8.05 (d, J=7.6 Hz, 2H), 7.82 (d, J=7.6 Hz, 2H), 7.57 (d, J=0.8 Hz, 1H), 7.47 (q, J=6.4 Hz, 3H), 7.39 (d, J=8.0 Hz, 2H), 7.32-7.27 (m, 5H), 7.23-7.07 (m, 27H), 6.99-6.96 (m, 5H), 6.94-6.84 (m, 3H); ¹³C NMR (100 MHz, CDCl₃) δ 152.6, 152.0, 150.5, 149.6, 147.7, 147.4, 145.4, 144.34, 144.29, 141.8, 141.4, 140.5, 139.1, 138.3, 137.9, 135.1, 134.5, 133.8, 132.2, 131.6, 129.8, 129.6, 129.3, 129.1, 128.5, 128.3, 128.1, 127.9, 127.46, 127.41, 127.2, 126.6, 124.2, 122.9, 122.8, 121.8, 121.2, 120.6, 120.5, 120.4, 120.1, 119.4, 110.4, 65.2, 65.0; TLC R_{f} 0.50 (CH₂Cl₂/hexanes, 1/1); High Resolution-MS calcd for C₇₈H₅₀N₂: 1014.3974, found: 1014.3974.

Evaluation of the Spirally Configured Cis-Stilbene/Fluorene Hybrid Material as Organic Luminescent Layer

The compounds of the present examples were evaluated for their thermal, photophysical, and electrochemical properties, such as their wavelengths of maximum absorpotion (Abs. λmax), wavelengths of maximum emission (Em, λmax), full width at half maximum (FWHM), quantum yield (Φ_{f}), oxidation potential (Eₒₓ), reduction potential (E_{red}), the highest occupied molecular orbital (E_{HOMO}), the lowest occupied molecular orbital (E_{LUMO}), energy gap (E_{g}), the glass transition temperature (T_{g}) and the decomposition temperature (T_{d}). Those properties mentioned above were measured according to the following methods and conditions.

The wavelengths of maximum absorpotion (Abs. λₘₐₓ), the wavelengths of maximum emission (Em, λₘₐₓ), and full width at half maximum (FWHM) were measured in a solution using dichloromethane as the solvent. Quantum yield (Φ_{f}) was measured with the spectrometer Hamamatsu C9920. The decomposition temperature was measured by a thermogravimetric analyzer (TGA). And the glass transition temperature was measured by differential scanning calorimeter (DSC).

The electrochemical properties, including Eₒₓ, E_{red}, E_{HOMO}, and E_{LUMO} were measured by way of cyclic voltammetry (CV) in a solution using dichloromethane or tetrahydrofuran as a solvent. Platinum wire electrode was used as a counter electrode and glassy carbon electrode was used as a working electrode and Ag/Ag⁺ as a reference electrode. Ferrocene was used as a standard. The CV curves were calibrated using the ferrocene/ferrocenium (Fc/Fc⁺) redox couple as an external standard which was measured under same condition before and after the measurement of samples. The energy level of Fc/Fc+ was assumed at −4.8 eV to vacuum. The energy level of LUMO (E_{LUMO}) was obtained by adding the bandgap energy (E_{g}) to the HOMO energy level (E_{HOMO}).

Measurement Data (Thermal Stabilities, Photophysical Properties and Electrochemical Properties) of the Compounds of Chemical Formula (1), Chemical Formula (2), Chemical Formula (3), Chemical Formula (4a) and Chemical Formula (5) are Shown in Table 1.

TABLE 1

| | | | | | Com- |
|---|---|---|---|---|---|
| | Compound | Compound | Compound | Compound | pound |
| | 1 | 2 | 3 | 4a | 5 |
| Abs. λₘₐₓ | 375 | 379 (38.9) | 416 | 389 | 416 |
| (εₘₐₓ × 10³)^{a} | (29.5) | | (46.4) | (44.6) | (25.3) |
| nm | | | | | |
| Em, λₘₐₓ^{a} | 421, 445 | 421, 445 | 475, 499 | 432, 460 | 533 |
| nm (fwhm)^{c} | (58) | (58) | (79) | (51) | (75) |
| Φ_{f},^{a}, % | 99 | 99 | 99 | 99 | 99 |
| Eₒₓ^{a} (V)/ | +0.81/ | +0.85/ | +0.43/ | +1.03/ | +0.33/ |
| E_{red}^{b} (V) | −2.09 | −2.13 | −2.13 | −1.77 | −1.84 |
| T_{g} (° C.)/ | N.D.^{e}/ | 256/ | 234/ | 259/ | 242/ |
| T_{d} (° C.) | 471 | 470 | 492 | 469 | 482 |
| ΔE | 2.90 | 2.98 | 2.56 | 2.80 | 2.17 |
| (E_{g}, eV)^{d} | (3.06) | (3.07) | (2.74) | (2.96) | (2.68) |

^{a}Measured in CH₂Cl₂.
^{b}Measured in THF.
^{c}was full width at half maximum (FWHM).
^{d}The bandgap of HOMO-LUMO, E_{g}, which was derived from the observed optical edge.
^{e}N.D: not detected.

Regarding to the thermal stabilities, according to Table 1, the decomposition temperatures of chemical formula (1), chemical formula (2), chemical formula (3), chemical formula (4a), and chemical formula (5) are higher than 469° C. Meanwhile, the glass transition temperatures of chemical formula (1), chemical formula (2), chemical formula (3), chemical formula (4a), and chemical formula (5) are higher than 234° C. Accordingly, the excellent thermal stabilities of the serials of the spirally configured cis-stilbene/fluorene hybrid compounds as disclosed above are attributed to the intra-molecular spiral configuration and the diindeno-fused templates. In summary, the chemical formula (1), chemical formula (2), chemical formula (3), chemical formula (4a), and chemical formula (5) are materials with high thermal stability.

Regarding to the electrochemical properties, both the compounds of the chemical formula (1) and chemical formula (2) have one set of quasi-reversible oxidation potentials at 0.83±0.02 V. They are attributed to that oxidation mainly occurs at the spiro fluorene fragments of the compounds. On the other hand, both the compounds of the chemical formula (1) and chemical formula (2) have reduction potentials at −2.11±0.02 V. They are attributed to the reduction mainly occurs at cis-stilbene fragment of the STIF template. The compound of the chemical formula (4a) has one set of irreversible oxidation potential at 1.03 V. It is attributed to that oxidation mainly occurs at the spiro fluorene fragment of the compound. Also, the compound of the chemical formula (4a) has one set of quasi-reversible reduction potential at −1.77 V. It is attributed to that reduction mainly occurs at cyano group of the compound. Both the compounds of the chemical formula (3) and chemical formula (5) have one set of quasi-reversible oxidation potentials between 0.33 and 0.43 V, which is resulted from the diphenyl amino groups of the compounds. The compound of the chemical formula (3) has one set of irreversible reduction potential at −2.13 V. It is attributed to that reduction occurs at the cis-stilbene fragment of STIF template in the compound. The compound of the chemical formula (5) has one set of irreversible reduction potential at −1.84 V. It is attributed to that reduction occurs at cyano group in the compound.

In addition, the absorption peaks which appear around 375 nm for both compounds of the chemical formula (1) and chemical formula (2) correspond to their HOMO-LUMO transitions, and their electronic clouds mainly distribute around dibenzocycloheptene template. The absorption peaks which appear around 320 nm for both compounds of the chemical formula (1) and chemical formula (2) correspond to their orthogonal intramolecular charge transfer between the spiro fluorene fragment and the dibenzocycloheptene moiety (HOMO−1→LUMO) and the π→π* absorption of spiro fluorene(HOMO−1→LUMO+1). As for the compound of the chemical formula (3), its absorption peak which appears at 416 nm corresponds to the HOMO-LUMO transition, and its distribution of electronic clouds switches to the central dibenzocycloheptene template from the conjugation system of the diphenyl amino substituents and the dibenzocycloheptene moiety. And its absorption peak which appears around 340 nm corresponds to the orthogonal intramolecular charge transfer between its spiro fluorene fragment and its dibenzocycloheptene moiety (HOMO−2→LUMO) and the charge transfer between its diphenyl amino substituents to its dibenzocycloheptene moiety (HOMO−1→LUMO). In addition, as for the compound of the chemical formula (4a), the absorption peak which appears around 389 nm corresponds to the HOMO-LUMO transition, and its distribution of electronic clouds switches to the conjugation system of the indeno moiety and the dibenzocycloheptene template from the central dibenzocycloheptene template. And its absorption peak which appears around 350 nm corresponds to the orthogonal intramolecular charge transfer between its spiro fluorene fragment to the whole upper panel which includes the two cyano groups (HOMO−1→LUMO) and the π→π*absorption of spiro fluorene (HOMO−1→LUMO+2). As for the compound of the chemical formula (5), its absorption peak which appears around 416 nm corresponds to the HOMO-LUMO transition and that appears around 310 nm corresponds to the orthogonal intramolecular charge transfer from its spiro fluorene fragment to the conjugation system between the cyano groups and the dibenzocycloheptene moiety (HOMO−2→LUMO) and the π→π* absorption of its spiro fluorine (HOMO−2→LUMO+2). Overall, the energy levels of HOMO and LUMO of chemical formula (1) to chemical formula (4a) are majorly contributed from the dibenzocycloheptene moiety. The optical emission of the compounds mainly appears at their STIF templates with confined cis-stilbene. Therefore, the serials of the spirally configured cis-stilbene/fluorene hybrid materials of the present invention have excellent luminous efficiency of confined cis-stilbene.

Moreover, both of the compounds of chemical formula (1) and chemical formula (2) emit bluish light and have double emission peaks at 421 nm and 445 nm, and their full widths at half maximum of the emission peaks are 58 nm, which represents both compounds can be excellent bluish electroluminescence materials. As for the compound of the chemical formula (3), because the dibenzocycloheptene moiety is an electron acceptor, the compound of chemical formula (3) has charge transfer from the diphenyl amino groups to the dibenzocycloheptene moiety. The compound of the chemical formula (3) is a bluish-green emitter whose emission peaks appear around 475 nm and 499 nm. The emission peaks of the compound of the chemical formula (4a), which is a sky-blue emitter, appear around 432 nm and 460 nm and are attributed to the elongated conjugation system of the whole molecule resulted from the cyano groups. The extended conjugated intramolecular excited state charge transfer of the chemical formula (5) results in that the compound has a greenish yellow luminescence with an emission peak at 533 nm. In addition, the quantum yield (Φ_{f}, %) of the series of compounds of the chemical formula (1), chemical formula (2), chemical formula (3), chemical formula (4a), and chemical formula (5) are all 99%, which represents these compounds have excellent luminous efficiency of confined cis-stilbene. The spiro fluorene fragment and the fused indeno moieties both increase the rigidity of the molecules greatly and thus reduce the possibilities of radiationless decay.

In addition, E_{HOMO} of the compounds of the chemical formula (1), chemical formula (2), chemical formula (3), chemical formula (4a), and chemical formula (5), range from −5.1 to −5.8 eV, and their E_{LUMO} range from −2.7 to −3.0 eV. Their E_{g} range from 2.17 to 2.98 eV.

The Device Efficiency for Compounds of Chemical Formula (1), Chemical Formula (2), Chemical Formula (3), Chemical Formula (4a), and Chemical Formula (5) which were Used in Organic Electroluminescent Device.

Herein, the devices to be tested (B1 to B24, BG1 to BG3, SB1 to SB3, and Y1 to Y4) were constructed with indium tin oxide (ITO) coated glass as the anode substrate, LiF/Al as the cathode, poly(3,4-ethylenedioxythiophene) polystyrene sulfonate (PEDOT:PSS) deposited on to the indium tin oxide (ITO) surface as the hole injection layer, optionally N,N-bis-(1-naphthyl)-N,N-diphenyl-1,1-biphenyl-4,4-diamine (NPB) or 1,1-Bis[4-[N,N′-di(p-tolyl)amino]phenyl]cyclohexane (TAPC) as the hole transport layer, 2,2′,2″-(1,3,5-benzinetriyl)-tris(1-phenyl-1-H-benzimidazole) (TPBI) or Bphen as the electron transport layer, and the compounds of the chemical formula (1), chemical formula (2), chemical formula (3), chemical formula (4a), and chemical formula (5) are used as the organic luminescent layer, hole transport layer, and/or the electron transport layer in the devices.

The configuration of each device is shown as follow. B1: ITO/NPB (40 nm)/chemical formula (1) (30 nm)/TPBI (40 nm)/LiF (1 nm)/Al; B2: ITO/NPB (40 nm)/chemical formula (1) (30 nm)/Bphen (40 nm)/LiF (1 nm)/Al; B3: ITO/TCTA (40 nm)/chemical formula (1) (30 nm)/TPBI (40 nm)/LiF (1 nm)/Al; B4: ITO/TCTA (40 nm)/chemical formula (1) (30 nm)/Bphen (40 nm)/LiF (1 nm)/Al; B5: ITO/NPB (40 nm)/chemical formula (1) (30 nm)/Bphen (20 nm)/LiF (1 nm)/Al B6: ITO/NPB (60 nm)/chemical formula (1) (30 nm)/Bphen (10 nm)/LiF (1 nm)/Al; B7: ITO/NPB (60 nm)/chemical formula (1) (30 nm)/Bphen (15 nm)/LiF (1 nm)/Al; B8: ITO/NPB (60 nm)/chemical formula (1) (30 nm)/Bphen (20 nm)/LiF (1 nm)/Al; B9: ITO/NPB (50 nm)/TCTA (10 nm)/chemical formula (1) (30 nm)/Bphen (15 nm)/LiF (1 nm)/Al; B10: ITO/NPB (50 nm)/TCTA (10 nm)/chemical formula (1) (30 nm)/Bphen (20 nm)/LiF (1 nm)/Al B11: ITO/TCTA (10 nm)/chemical formula (1) (30 nm)/TPBI (40 nm)/LiF (1 nm)/Al B12: ITO/TCTA (20 nm)/chemical formula (1) (30 nm)/TPBI (40 nm)/LiF (1 nm)/Al; B13: ITO/TCTA (10 nm)/chemical formula (1) (40 nm)/TPBI (40 nm)/LiF (1 nm)/Al; B14: ITO/TCTA (20 nm)/chemical formula (1) (40 nm)/TPBI (40 nm)/LiF (1 nm)/Al B15: ITO/TCTA (30 nm)/chemical formula (1) (40 nm)/TPBI (40 nm)/LiF (1 nm)/Al; B16: ITO/chemical formula (1) (80 nm)/LiF (1 nm)/Al; B17: ITO/PEDOT/chemical formula (1) (80 nm)/LiF (1 nm)/Al B18: ITO/PEDOT/TCTA (40 nm)/chemical formula (1) (40 nm)/LiF (1 nm)/Al B19: ITO/PEDOT/chemical formula (1) (40 nm)/TPBI (40 nm)/LiF (1 nm)/Al; B20: ITO/PEDOT/TCTA (20 nm)/chemical formula (1) (30 nm)/TPBI (40 nm)/LiF (1 nm)/Al; B21: ITO/PEDOT/chemical formula (2) (80 nm)/LiF (1 nm)/Al; B22: ITO/PEDOT/TCTA (40 nm)/chemical formula (2) (40 nm)/LiF (1 nm)/Al; B23: ITO/PEDOT/chemical formula (2) (40 nm)/TPBI (40 nm)/LiF (1 nm)/Al; B24: ITO/PEDOT/TCTA (20 nm)/chemical formula (2) (30 nm)/TPBI (40 nm)/LiF (1 nm)/Al∘BG1: ITO/PEDOT/chemical formula (3) (80 nm)/LiF (1 nm)/Al; BG2: ITO/PEDOT/chemical formula (3) (40 nm)/TPBI (40 nm)/LiF (1 nm)/Al; BG3:ITO/PEDOT/TCTA (20 nm)/chemical formula (3) (40 nm)/TPBI (40 nm)/LiF (1 nm)/Al∘SB1: ITO/PEDOT/chemical formula (4a) (80 nm)/LiF (1 nm)/Al; SB2: ITO/PEDOT/TCTA (40 nm)/chemical formula (4a) (40 nm)/LiF (1 nm)/Al; SB3:ITO/PEDOT/TCTA (20 nm)/chemical formula (4a) (30 nm)/TPBI (40 nm)/LiF (1 nm)/Al; Y1: ITO/PEDOT/chemical formula (5) (80 nm)/LiF (1 nm)/Al∘Y2: ITO/PEDOT/NPB (40 nm)/chemical formula (5) (40 nm)/LiF (1 nm)/Al; Y3: ITO/PEDOT/chemical formula (5) (40 nm)/TPBI (40 nm)/LiF (1 nm)/Al; Y4: ITO/PEDOT/NPB (40 nm)/chemical formula (5) (40 nm)/TPBI (40 nm)/LiF (1 nm)/Al∘

The obtained efficiencies of each organic electroluminescent device are shown as in Table 2.

TABLE 2

| | Em. λₘₐₓ,^{a} | | | | | η_{c}^{b}/ηₚ^{b} | Lₘₐₓ (L₂₀)^{b} | | |
|---|---|---|---|---|---|---|---|---|---|
| Device # | (nm) | | Vₒₙ^{b} (V) | | ηₑₓₜ^{b} (%) | (cd/A)/(lm/W) | (cd/m²) | | CIE 1931 |
| B1 | 454 | (73) | 4.9 | (8.8) | 1.29 | 1.34/0.48 | 2230 | (267) | (0.18, 0.16) |
| B2 | 456 | (76) | 2.9 | (5.2) | 3.73 | 3.87/2.36 | 4378 | (767) | (0.28, 0.22) |
| B3 | 432 | (69) | 5.9 | (8.1) | 2.17 | 2.25/0.88 | 1883 | (444) | (0.17, 0.13) |
| B4 | 456 | (75) | 4.9 | (7.2) | 2.38 | 2.47/1.09 | 862 | (484) | (0.16, 0.15) |
| B5 | 456 | (81) | 2.5 | (4.1) | 1.69 | 1.76/1.37 | 2020 | (347) | (0.17, 0.15) |
| B6 | 432 | (75) | 2.5 | (4.3) | 1.87 | 1.94/1.44 | 2456 | (383) | (0.16, 0.13) |
| B7 | 432 | (74) | 2.5 | (4.3) | 2.11 | 2.19/1.60 | 2571 | (430) | (0.16, 0.13) |
| B8 | 432 | (70) | 2.5 | (4.1) | 1.72 | 1.79/1.37 | 2012 | (352) | (0.17, 0.13) |
| B9 | 432 | (68) | 2.7 | (4.5) | 1.34 | 1.40/0.97 | 1397 | (279) | (0.18, 0.15) |
| B10 | 432 | (65) | 2.7 | (4.5) | 1.74 | 1.81/1.26 | 1453 | (361) | (0.18, 0.14) |
| B11 | 456 | (83) | 4.3 | (7.2) | 1.81 | 1.88/0.83 | 1855 | (370) | (0.17, 0.17) |
| B12 | 456 | (89) | 4.8 | (6.4) | 2.62 | 2.72/1.34 | 3058 | (529) | (0.16, 0.16) |
| B13 | 456 | (78) | 4.8 | (7.2) | 2.43 | 2.52/1.10 | 2265 | (496) | (0.17, 0.17) |
| B14 | 456 | (72) | 6.0 | (8.2) | 2.41 | 2.51/0.96 | 2011 | (497) | (0.17, 0.14) |
| B15 | 456 | (67) | 6.1 | (8.7) | 2.74 | 2.85/1.03 | 1726 | (561) | (0.17, 0.13) |
| B16 | 564 | (214) | 12.6 | (15.3) | 0.02 | 0.02/0.004 | 9 | (4) | (0.319, 0.347) |
| B17 | 460 | (69) | 8.4 | (10.7) | 0.14 | 0.14/0.04 | 226 | (28) | (0.176, 0.190) |
| B18 | 432 | (72) | 4.9 | (7.2) | 0.27 | 0.29/0.13 | 495 | (56) | (0.167, 0.138) |
| B19 | 456 | (73) | 4.5 | (5.6) | 2.24 | 2.32/1.31 | 3524 | (457) | (0.165, 0.140) |
| B20 | 454 | (72) | 4.0 | (6.5) | 2.73 | 2.84/1.37 | 4049 | (565) | (0.162, 0.138) |
| B21 | 456 | (78) | 6.5 | (7.5) | 0.12 | 0.12/0.05 | 90 | (25) | (0.17, 0.15) |
| B22 | 432 | (74) | 5.3 | (6.3) | 0.2 | 0.21/0.10 | 277 | (43) | (0.16, 0.12) |
| B23 | 454 | (75) | 4.1 | (5.5) | 1.92 | 1.99/1.13 | 3467 | (397) | (0.16, 0.13) |
| B24 | 456 | (74) | 3.3 | (5.9) | 2.41 | 2.50/1.33 | 4352 | (499) | (0.16, 0.13) |
| BG1 | 480 | (88) | 7.5 | (8.9) | 0.03 | 0.03/0.01 | 104 | (6) | (0.25, 0.43) |
| BG2 | 482 | (77) | 4.9 | (7.5) | 4.08 | 4.23/1.77 | 8435 | (847) | (0.23, 0.46) |
| BG3 | 480 | (73) | 4.5 | (8.0) | 3.35 | 3.48/1.37 | 9482 | (696) | (0.22, 0.45) |
| SB1 | 470 | (72) | 5.6 | (8.1) | 0.08 | 0.09/0.03 | 757 | (17) | (0.18, 0.22) |
| SB2 | 470 | (81) | 3.8 | (6.0) | 3.24 | 3.37/1.76 | 6876 | (673) | (0.18, 0.22) |
| SB3 | 472 | (88) | 6.0 | (8.4) | 2.55 | 2.65/0.99 | 3862 | (537) | (0.19, 0.28) |
| Y1 | 528 | (115) | 4.9 | (9.7) | 1.57 | 1.63/0.53 | 5289 | (326) | (0.37, 0.56) |
| Y2 | 530 | (107) | 2.8 | (7.4) | 3.44 | 3.57/1.52 | 8285 | (711) | (0.35, 0.57) |
| Y3 | 528 | (105) | 4.2 | (7.3) | 5.22 | 5.42/2.33 | 13990 | (1076) | (0.35, 0.57) |
| Y4 | 520 | (95) | 4.2 | (7.6) | 5.12 | 5.32/2.11 | 11060 | (1063) | (0.34, 0.58) |

^{a}The numbers in the brackets represent full width at half maximum (FWHM, nm) of their corresponding Em. λₘₐₓ.
^{b}Vₒₙ: turn-on voltage (V); ηₑₓₜ: external quantum efficiency (%); η_{c}: current efficiency (cd/A); ηₚ: power efficiency (lm/W); L: luminance (cd/m²).
Vₒₙ, ηₑₓₜ, η_{c}, ηₚ, and L₂₀ (i.e., the values in the brackets of the luminance column) are measured at 20 mA/cm².

As shown in the measuring results listed above, the thickness of electron transport layer in device B5 was half of that in device B2, and the CIE_{xy} coordinates of the device B5 thus returned to the range of ordinary blue light from that of device B2 (0.28, 0.22), which has dual emittion bands. Therefore, we can know that the recombination zone of device B2 was located at the juncture between its organic luminescent layer and its electron transport layer, which resulted in dual emission peaks. The device B5 has a shortened electron transport distance, so that the electrons may enter to the organic luminescent layer faster and the carriers may also recombine in the organic luminescent layer, which helps to reduce the occurrence of exciplexes (hetero-excimers).

On the other hand, when comparing to the device B12 which did not have a PEDOT hole injection layer, the device B20 with the PEDOT hole injection layer had a slightly improved luminous efficiency, and its bluish emitting light became deeper (CIEₓ˜0.15, CIE_{y}<0.15). In addition, the efficiency roff-off under currents of high densities was also suppressed. These results represent that PEDOT actually helps to improve the efficiency of hole injection.

As shown in Table 2, the obtained efficiencies of the devices B18 and B19 validated that the compound of the chemical formula (1) had a higher hole transport rate. Therefore, the device B19, in which the compound of chemical formula (1) functioned as the hole transport layer and the organic luminescent layer, had an excellent luminous efficiency. At 20 mA/cm², ηₑₓₜ, η_{c}, and ηₚ of the device B19 were 2.2%, 2.3 cd/A, and 1.3 lm/W, respectively.

In the devices with similar configurations, those devices with the compounds of chemical formula (2), except the device 22, have lower working voltages at 20 mA/cm² and lower Vₒₙ than those with the compounds of chemical formula (1). It might be attributed to that the transport property of the compound of the chemical formula (2) was better than that of the compound of the chemical formula (1). Therefore, the compounds of the chemical formula (2) can be doped in other host materials so as to reduce the concentration quenching effect caused by self-quenching and self-absorption.

The device BG2, which used the compound of the chemical formula (3) as both hole transport layer and organic luminescent layer, at 20 mA/cm², had a ηₑₓₜ of 4.1%, a η_{c} of 4.2 cd/A, a ηₚ of 1.8 lm/W and a luminance of 847 cd/m², and emitted a bluish green light with a CIE_{xy} coordinate of (0.23, 0.46). When appended with the substituents of diphenyl amino groups, the hole transport rate of the compounds of chemical formula (3) was increased. Therefore, the compounds of chemical formula (3) are suitable to be used as a material for both the hole transport layer and the organic luminescent layer.

The device SB2, which used the compounds of the chemical formula (4a) as both electron transport layer and organic luminescent layer, also had a good performance. At 20 mA/cm², the ηₑₓₜ, η_{c}, ηₚ, and luminance of the device SB2 were 3.2%, 3.4 cd/A, 1.8 lm/W, and 673 cd/m², respectively. It emitted a sky-blue light with a CIE_{xy} coordinate of (0.18, 0.22).

The excellent performance of the single-layered device Y1 represents that the compound of the chemical formula (5) is an ambipolar molecule and has a good balance of carrier transport. At 20 mA/cm², the ηₑₓₜ, η_{c}, and ηₚ of the device Y1 were 1.6%, 1.6 cd/A, and 0.5 lm/W, respectively. The device Y1 emitted a green light with a CIE_{xy} coordinate of (0.37, 0.56). The device Y2, which used NPB as the hole transport layer based on the matching of energy level to the compound of the chemical formula (5), had improved and better performance two times more than that of the device Y1. Moreover, the performance of the device Y3 was three times more than that of the device Y1. At 20 mA/cm², the ηₑₓₜ of the device Y3 was 5.2%, which was beyond the maximal theoretical value. Also, the η_{c} and ηₚ of the device Y3 were 5.4 cd/A and 2.3 lm/W, respectively. The performance of the triple-layered device Y4 was slightly lower than that of the device Y3. However, at 20 mA/cm², the ηₑₓₜ of the device Y4 also exceeded the maximal theoretical value and reach to 5.1%, and the η_{c} and ηₚ of the device Y4 were 5.3 cd/A and 2.1 lm/W, respectively. Those results discussed as above represented that the compound of the chemical formula (5) had a higher rate of hole transport than the rate of electron transport. Therefore, because the compound of the chemical formula (5) has indeno groups, which has hole transport properties, its balance of carrier transport is therefore improved. Accordingly, the device Y1 has an excellent performance.

Accordingly, the series of the spirally configured cis-stilbene/fluorene hybrid materials according to the embodiments shown as above are suitable to be applied in the organic luminescent layer of an organic luminescent device, and they are also suitable to be applied in the layer with both hole transport and organic luminescent properties, or be applied in the layer with both electron transport and organic luminescent properties.

As mentioned above, in the spirally configured cis-stilbene/fluorene hybrid material and the organic electroluminescent device by using the same according to some embodiments of the present invention, the confined cis-stilbene fragment and the fluorine are doubly ortho-linked at C9 position, such that the cis-stilbene fragment is fixed and prevented from isomerization. In addition, two indeno templates are fused with the spirally configured cis-stilbene/fluorene hybrid system at C2-C3 and C7-C8 positions, and the resulting compound has a sterically-hindered structure so as to prevent the fluorescence quenching effect caused by π-π stacking. Moreover, when the confined cis-stilbene, which has high fluorescence quantum effect, is incorporated with the spirally configured fluorene and diindeno-fused system, the thermal stability and the film forming ability of the whole material can be further increased. Therefore, the resulting spirally configured cis-stilbene/fluorene hybrid material is suitable for an organic electroluminescent device.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A spirally configured cis-stilbene/fluorene hybrid material, comprising a structure of the following General Formula (1), 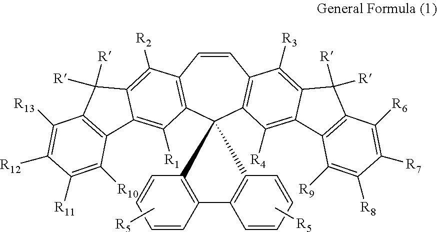
wherein, R′ is an alkyl group or represented by General Formula (2), 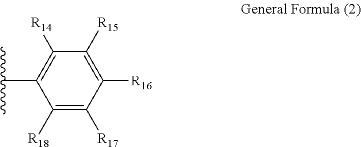
wherein, R₁ to R₄, R₆, R₈ to R₁₁ and R₁₃ to R₁₈ are each independently selected from the group consisting of a hydrogen atom, halogen atom, cyano group, alkyl group, cycloalkyl group, alkoxy group, amino group, haloalkyl group, thioalkyl group, silyl group and alkenyl group, R₅ is a hydrogen atom, tert-butyl group or aryl group, and R₇ and R₁₂ are each independently selected from the group consisting of a hydrogen atom, aryl group, diarylamine, cyano group and hetero aromatic ring.

2. The spirally configured cis-stilbene/fluorene hybrid material of claim 1, wherein the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the secondary amino group is an amino group having one aromatic ring substituent or having one C1-C6 straight-chain alkyl substituent, branch-chain alkyl substituent, or non-aromatic ring substituent, the tertiary amino group is an amino group having two independent aromatic ring substituents or having two independent C1-C6 straight-chain alkyl substituent, branch-chain alkyl substituent, or non-aromatic ring substituent, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

3. The spirally configured cis-stilbene/fluorene hybrid material of claim 1, being represented by following chemical formula (1), chemical formula (2), chemical formula (3), chemical formula (4a), chemical formula (4b), or chemical formula (5).

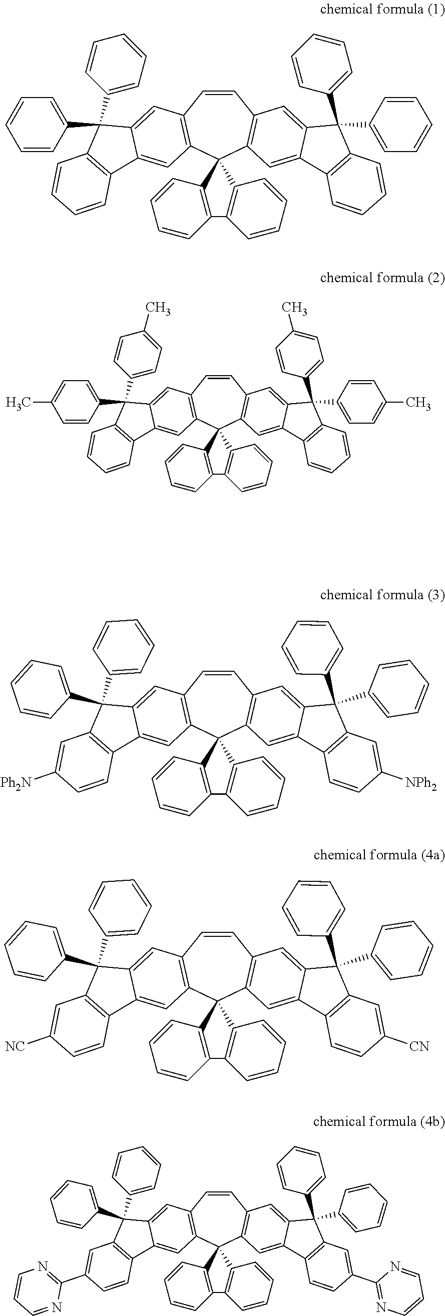

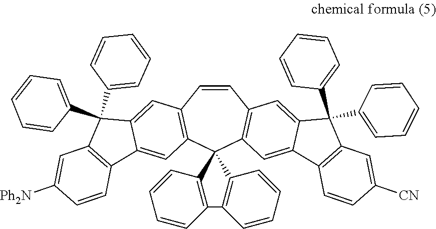

4. The spirally configured cis-stilbene/fluorene hybrid material of claim 1, having glass transition temperatures ranged from 234° C. to 259° C.

5. The spirally configured cis-stilbene/fluorene hybrid material of claim 1, having decomposition temperatures ranged from 469° C. to 492° C.

6. The spirally configured cis-stilbene/fluorene hybrid material of claim 1, having oxidation potentials ranged from 0.33V to 1.03V and reduction potentials ranged from −1.77V to −2.13V.

7. The spirally configured cis-stilbene/fluorene hybrid material of claim 1, having highest occupied molecular orbital energy levels (E_{HOMO}) ranged from −5.1 eV to −5.8 eV and lowest unoccupied molecular orbital energy levels (E_{LUMO}) ranged from −2.7 eV to −3.0 eV.

8. An organic electroluminescent device, comprising:
a first electrode layer;
a second electrode layer; and
an organic luminescent unit, deposited between the first electrode layer and the second electrode layer, wherein the organic luminescent unit has at least a spirally configured cis-stilbene/fluorene hybrid material as shown in General Formula (1), 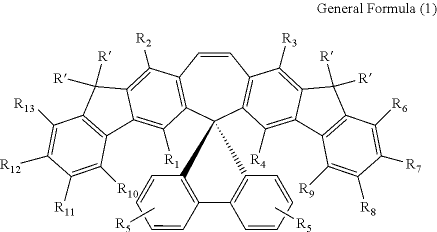
wherein, R′ is an alkyl group or represented by General Formula (2), 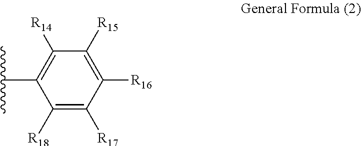
wherein, R₁ to R₄, R₆, R₈ to R₁₁ and R₁₃ to R₁₈ are each independently selected from the group consisting of a hydrogen atom, halogen atom, cyano group, alkyl group, cycloalkyl group, alkoxy group, amino group, haloalkyl group, thioalkyl group, silyl group and alkenyl group, R₅ is a hydrogen atom, tert-butyl group or aryl group, and R₇ and R₁₂ are each independently selected from the group consisting of a hydrogen atom, aryl group, diarylamine, cyano group and hetero aromatic ring.

9. The organic electroluminescent device of claim 8, wherein the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the secondary amino group is an amino group having one aromatic ring substituent or having one C1-C6 straight-chain alkyl substituent, branch-chain alkyl substituent, or non-aromatic ring substituent, the tertiary amino group is an amino group having two independent aromatic ring substituents or having two independent C1-C6 straight-chain alkyl substituent, branch-chain alkyl substituent, or non-aromatic ring substituent, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

10. The organic electroluminescent device of claim 8, wherein the spirally configured cis-stilbene/fluorene hybrid material has a structure represented by following chemical formula (1), chemical formula (2), chemical formula (3), chemical formula (4a), chemical formula (4b), or chemical formula (5).

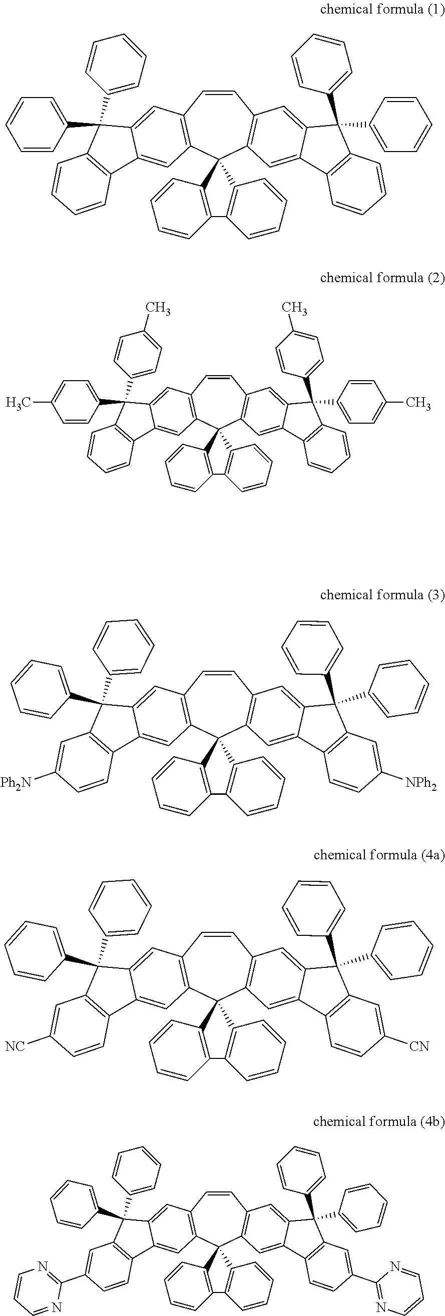

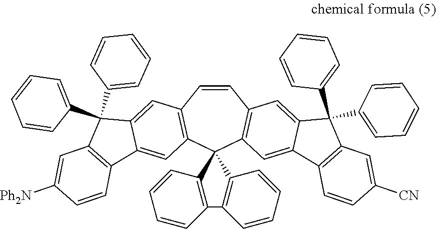

11. The organic electroluminescent device of claim 8, wherein the organic luminescent unit comprises an organic luminescent layer.

12. The organic electroluminescent device of claim 11, wherein the organic luminescent unit further comprises a hole transport layer and an electron transport layer, and the organic luminescent layer is deposited between the hole transport layer and the electron transport layer.

13. The organic electroluminescent device of claim 11, wherein the organic luminescent unit further comprises a hole injection layer, a hole transport layer, an electron transport layer and an electron injection layer, and the hole transport layer, the organic luminescent layer and the electron transport layer are sequentially deposited between the hole injection layer and the electron injection layer.

14. The organic electroluminescent device of claim 11, wherein the organic luminescent layer comprises the spirally configured cis-stilbene/fluorene hybrid material.

15. The organic electroluminescent device of claim 11, wherein the organic luminescent layer comprises a host material and a guest material, and the host material comprises the spirally configured cis-stilbene/fluorene hybrid material.
